(19) United States
(12) Patent Application Publication
(10) Pub. No.: US 2018/0251836 A1
(43) Pub. Date: Sep. 6, 2018

(54) NOVEL MIRNA BIOMARKERS AND USE THEREOF

(71) Applicant: HUMMINGBIRD DIAGNOSTICS GMBH, Heidelberg (DE)

(72) Inventors: Christina Backes, Saarbrucken-Dudweiler (DE); Andreas Keller, Puttlingen (DE); Eckart Meese, Huetschenhausen (DE)

(21) Appl. No.: 15/755,481

(22) PCT Filed: Aug. 30, 2016
(86) PCT No.: PCT/EP2016/070407

§ 371 Date: Feb. 26, 2018

(30) Foreign Application Priority Data
Sep. 2, 2015 (EP) 15183409.0

(51) Int. Cl. C12Q 1/6883; C12Q 1/6865

(52) U.S. Cl. CPC C12Q 1/6883; C12Q 1/6865; C12Q 2600/178; C12Q 2600/158; C12Q 2600/118

(57) ABSTRACT

The present invention relates to novel isolated nucleic acid molecules (novel miRNAs and novel miRNA precursor molecules) as well as vectors, host cells, primers, cDNA-transcripts, polynucleotides derived from said isolated nucleic acid molecules and their use in diagnosis and therapy. Furthermore the present invention relates to methods and kits for diagnosing a disease, such as Multiple Sclerosis (MS) or Alzheimer's Disease (AD) employing said novel isolated nucleic acid molecules (novel miRNAs molecules).

### TECHNICAL FIELD OF THE INVENTION

The present invention relates to novel isolated nucleic acid molecules (novel miRNAs and novel miRNA precursor molecules) as well as vectors, host cells, primers, cDNA-transcripts, polynucleotides derived from said isolated nucleic acid molecules and their use in diagnosis and therapy. Furthermore the present invention relates to methods and kits for diagnosing a disease, such as Multiple Sclerosis (MS) or Alzheimer's Disease (AD) employing said novel isolated nucleic acid molecules (novel miRNAs molecules).

### BACKGROUND OF THE INVENTION

MicroRNAs (miRNAs) are a new class of biomarkers. They represent a group of small noncoding RNAs that regulate gene expression at the posttranslational level by degrading or blocking translation of messenger RNA (mRNA) targets. MiRNAs are important players when it comes to regulate cellular functions and in several diseases, including cancer or neurodegenerative diseases.

So far, miRNAs have been extensively studied in tissue material. It has been found that miRNAs are expressed in a highly tissue-specific manner. Disease-specific expression of miRNAs have been reported in many human cancers employing primarily tissue material as the miRNA source. Since recently it is known that miRNAs are not only present in tissues but also in other body fluid samples, including human blood.

In order to improve the biomarker capabilities in diagnosis, there is a constant need for disease specific, well-performing biomarkers such as miRNA biomarkers. The inventors of the present invention addressed the identification of novel miRNAs from blood samples. By combining a Next Generation Sequencing workflow with an innovative biostatistics pipeline, the inventors were able to identify a set of 37 novel miRNA molecules and validated the identity of said miRNAs by qRT-PCT and a cloning approach. Surprisingly, said set of 37 novel miRNAs proved to be differentially regulated between healthy control subjects and disease subjects, such as Multiple Sclerosis (MS) and/or Alzheimer Disease (AD) subjects. Thus, said novel miRNAs are suitable for use in diagnosis and/or prognosis of diseases, such as Multiple Sclerosis (MS) and/or Alzheimer's Disease (AD).

### SUMMARY OF THE INVENTION

In a first aspect, the invention provides an isolated nucleic acid molecule comprising a nucleotide sequence presented as SEQ ID NO: 1-37, a fragment thereof, or a nucleotide sequence with at least 90%, 94%, 96% or greater sequence identity thereto.

In a second aspect, the invention provides an isolated nucleic molecule that is a complement to nucleic acid molecules according to the first aspect of the invention.

In a third aspect, the invention provides a vector comprising an isolated nucleic acid molecule according to the first or the second aspect of the invention.

In a fourth aspect, the invention provides a host cell transformed with the isolated nucleic acid molecules according to the first or second aspect of the invention.

In a fifth aspect, the invention provides a host cell transformed with the vector according to the third aspect of the invention.

In a sixth aspect, the invention provides a primer for reverse transcribing an isolated nucleic acid molecule of the first aspect of the invention.

In a seventh aspect, the invention provides a cDNA-transcript of an isolated nucleic acid molecule of the first aspect of the invention.

In an eighth aspect, the invention provides a set of primer pairs amplifying said cDNA-transcripts of the seventh aspect of the invention.

In a ninth aspect, the invention provides a polynucleotide for detecting an isolated nucleic acid molecule of the first or second aspect of the invention.

In a tenth aspect, the invention provides a cDNA-transcript according to the seventh aspect of the invention, hybridized to an isolated nucleic acid molecule of the first aspect of the invention.

In an eleventh aspect, the invention provides an isolated nucleic acid molecules according to the first aspect of the invention for use in diagnosis and/or prognosis of a disease or the invention provides the (in vitro) use of an isolated nucleic acid molecule of the first aspect of the invention for diagnosis and/or prognosis of a disease.

In a twelfth aspect, the invention provides an isolated nucleic acid molecules according to the first aspect of the invention for use as a medicament or the invention provides the (in vitro) use of an isolated nucleic acid molecules according to the first aspect of the invention for therapeutic intervention (therapy).

In a thirteenth aspect, the present invention provides a method for diagnosing and/or prognosing of a disease, comprising the steps:
(i) determining an expression profile of a set comprising at least one nucleic acid molecule of the first aspect of the invention, wherein said nucleic acid molecule is differentially expressed in the disease in a blood sample from a subject, and
(ii) comparing said expression profile to a reference, wherein the comparison of said expression profile to said reference allows for the diagnosis and/or prognosis of the disease.

In a fourteenth aspect, the present invention provides means for determining the expression of at least one isolated nucleic acid molecule of the first aspect of the invention, comprising
(a) one or more polynucleotides according to the ninth aspect of the invention, and
(b) a biochip, a RT-PCT system, a PCR-system, a flow cytometer, a Luminex system or a next generation sequencing system.

In a fifteenth aspect, the present invention provides a kit for diagnosing and/or prognosing a disease, comprising:
(a) means for determining the expression profile occording to the fourteenth aspect of the invention
(b) one or more reference expression profiles

This summary of the invention does not necessarily describe all features of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in this specification and in the appended claims, the singular forms “a”, “an”, and “the” include plural referents, unless the content clearly dictates otherwise. For example, the term “a test compound” also includes “test compounds”.

The terms “microRNA” or “miRNA” refer to single-stranded RNA molecules of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNAs are first transcribed as primary transcripts or pri-miRNAs with a cap and poly-A tail and processed to short, 70 nucleotide stem-loop structures known as pre-miRNAs in the cell nucleus. This processing is performed in animals by a protein complex known as the Microprocessor complex consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC. This strand is known as the guide strand and is selected by the argonaute protein, the catalytically active RNase in the RISC, on the basis of the stability of the 5′ end. The remaining strand, known as the miRNA*, anti-guide (anti-strand), or passenger strand, is degraded as a RISC substrate. Therefore, the miRNA*s are derived from the same hairpin structure like the “normal” miRNAs. So if the “normal” miRNA is then later called the “mature miRNA” or “guide strand”, the miRNA* is the “anti-guide strand” or “passenger strand”.

The terms “microRNA*” or “miRNA*” refer to single-stranded RNA molecules of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The “miRNA*s”, also known as the “anti-guide strands” or “passenger strands”, are mostly complementary to the “mature miRNAs” or “guide strands”, but have usually single-stranded overhangs on each end. There are usually one or more mispairs and there are sometimes extra or missing bases causing single-stranded “bubbles”. The miRNA*s are likely to act in a regulatory fashion as the miRNAs (see also above). In the context of the present invention, the terms “miRNA” and “miRNA*” are interchangeable used. The present invention encompasses (target) miRNAs which are dysregulated in biological samples such as blood of a diseased subject, preferably a AD and/or a MS subject in comparison to healthy controls. Said (target) miRNAs are preferably selected from the group consisting of SEQ ID NO: 1 to 37.

The term “miRBase” refers to a well established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download.

As used herein, the term “nucleotides” refers to structural components, or building blocks, of DNA and RNA. Nucleotides consist of a base (one of four chemicals: adenine, thymine, guanine, and cytosine) plus a molecule of sugar and one of phosphoric acid. The term “nucleosides” refers to glycosylamine consisting of a nucleobase (often referred to simply base) bound to a ribose or deoxyribose sugar. Examples of nucleosides include cytidine, uridine, adenosine, guanosine, thymidine and inosine. Nucleosides can be phosphorylated by specific kinases in the cell on the sugar's primary alcohol group (—CH2-OH), producing nucleotides, which are the molecular building blocks of DNA and RNA.

The term “polynucleotide”, as used herein, means a molecule of at least 10 nucleotides and of not more than 80 nucleotides covalently linked together. Preferably, the polynucleotides of the present invention are molecules of 10 to 70 nucleotides or 15 to 68 nucleotides in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67 or 68 nucleotides in length, not including optionally spacer elements and/or elongation elements described below. The depiction of a single strand of a polynucleotide also defines the sequence of the complementary strand. Polynucleotides may be single stranded or double stranded, or may contain portions of both double stranded and single stranded sequences.

The term “polynucleotide” means a polymer of deoxyribonucleotide or ribonucleotide bases and includes DNA and RNA molecules, both sense and anti-sense strands. In detail, the polynucleotide may be DNA, both cDNA and genomic DNA, RNA, cRNA or a hybrid, where the polynucleotide sequence may contain combinations of deoxyribonucleotide or ribonucleotide bases, and combinations of bases including uracil, adenine, thymine, cytosine, guanine, inosine, xanthine, hypoxanthine, isocytosine and isoguanine. Polynucleotides may be obtained by chemical synthesis methods or by recombinant methods.

In the context of the present invention, a polynucleotide as a single polynucleotide strand provides a probe (e.g. miRNA capture probe) that is capable of binding to, hybridizing with, or detecting a target of complementary sequence, such as a nucleotide sequence of a miRNA or miRNA*, through one or more types of chemical bonds, usually through complementary base pairing, usually through hydrogen bond formation. Polynucleotides in their function as probes may bind target sequences, such as nucleotide sequences of miRNAs or miRNAs*, lacking complete complementarity with the polynucleotide sequences depending upon the stringency of the hybridization condition. There may be any number of base pair mismatches which will interfere with hybridization between the target sequence, such as a nucleotide sequence of a miRNA or miRNA*, and the single stranded polynucleotide described herein. However, if the number of mutations is so great that no hybridization can occur under even the least stringent hybridization conditions, the sequences are no complementary sequences. The present invention encompasses polynucleotides in form of single polynucleotide strands as probes for binding to, hybridizing with or detecting complementary sequences of (target) miRNAs, that may be used in diagnosing and/or prognosing of a disease, preferably MS or AD. Said (target) miRNAs are preferably selected from the group consisting of SEQ ID NO: 1 to 37, more preferably selected from. SEQ ID NO: 3, 5, 28, 23, 10, 30, 27, 35, 33, 19, 14, 21, 31, 37, 29, 7, 32, 24 and 22 for diagnosing and/or prognosing Multiple Sclerosis, or are selected from SEQ ID NO: 28, 14, 2, 11, 36, 24, 34, 22, 19, 12, 8, 13, 32, 26, 15, 10, 21, 18, 6, 17 and 2 for diagnosing and/or prognosing Alzheimer's Disease.

The term “complement of a nucleic acid molecule”, as used in the context of the present invention, refers to sequences that are complementary to the nucleotide sequence of a novel isolated nucleotide molecule with SEQ ID NO: 1-37 according to the first aspect of the invention. In the context of the present invention, the terms “complement of a nucleic acid molecule” and “reverse complement of a nucleic acid molecule” are interchangeable used. Furthermore, it includes both complementary (and reverse complementary) DNA- and RNA-sequences. For example,e complements of the nucleic acid molecule novel-miR-1005 (SEQ ID NO: 1) with nucleotide sequence 5′-auucgcugggaauucagccucu-3′ (RNA) include the following:

| | |
|---|---|
| uaagcgaccuuaagucggaga | (complement, RNA) |
| TAAGCGACCTTAAGTCGGAGA | (complement, DNA) |
| agaggctgaattcccagcgaat | (reverse complement, RNA) |
| AGAGGCTGAATTCCAGCGAAT | (reverse complement, DNA) |

The term “blood sample”, as used in the context of the present invention, refers to a blood sample originating from a subject. The “blood sample” may be derived by removing blood from a subject by conventional blood collecting techniques, but may also be provided by using previously isolated and/or stored blood samples. For example a blood sample may be whole blood, plasma, serum, PBMC (peripheral blood mononuclear cells), blood cellular fractions including red blood cells (erythrocytes), white blood cells (leukocytes), platelets (thrombocytes), or blood collected in blood collection tubes (e.g. EDTA-, heparin-, citrate-, PAXgene-, Tempus-tubes) including components or fractions thereof. For example, a blood sample may be taken from a subject suspected to be affected or to be suspected to be affected by a disease, preferably AD and/or MS, prior to initiation of a therapeutic treatment, during the therapeutic treatment and/or after the therapeutic treatment.

Preferably, the blood sample from a subject (e.g. human or animal) has a volume of between 0.1 and 20 ml, more preferably of between 0.5 and 10 ml, more preferably between 1 and 8 ml and most preferably between 2 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

Preferably, when the blood sample is collected from the subject the RNA-fraction, especially the the miRNA fraction, is guarded against degradation. For this purpose special collection tubes (e.g. PAXgene RNA tubes from Preanalytix, Tempus Blood RNA tubes from Applied Biosystems) already including additives or additives that are added separately to the blood sample (e.g. RNAlater from Ambion, RNAsin from Promega) that stabilize the RNA fraction and/or the miRNA fraction are employed.

The term “biomarker”, as used in the context of the present invention, represents a characteristic that can be objectively measured and evaluated as an indicator of normal and disease processes or pharmacological responses. A biomarker is a parameter that can be used to measure the onset or the progress of disease or the effects of treatment. The parameter can be chemical, physical or biological.

The term “diagnosis” as used in the context of the present invention refers to the process of determining a possible disease or disorder and therefore is a process attempting to define the (clinical) condition of a subject. The determination of the expression level of a set of miRNAs according to the present invention correlates with the (clinical) condition of a subject. Preferably, the diagnosis comprises (i) determining the occurrence/presence of a disease, preferably AD and/or MS, (ii) monitoring the course of a disease, preferably AD and/or MS, (iii) staging of a disease, preferably AD and/or MS, (iv) measuring the response of a patient with a disease, preferably AD and/or MS to therapeutic intervention, and/or (v) segmentation of a subject suffering from a disease, preferably AD and/or MS.

The term “prognosis” as used in the context of the present invention refers to describing the likelihood of the outcome or course of a disease or a disorder. Preferably, the prognosis comprises (i) identifying of a subject who has a risk to develop a disease, preferably AD and/or MS, (ii) predicting/estimating the occurrence, preferably the severity of occurrence of a disease, preferably AD and/or MS, and/or (iii) predicting the response of a subject with a disease, preferably AD and/or MS to therapeutic intervention.

The term “miRNA expression profile” as used in the context of the present invention, represents the determination of the miRNA expression level or a measure that correlates with the miRNA expression level in a biological sample. The miRNA expression profile may be generated by any convenient means, e.g. nucleic acid hybridization (e.g. to a microarray, bead-based methods), nucleic acid amplification (PCR, RT-PCR, qRT-PCR, high-throughput RT-PCR), ELISA for quantitation, next generation sequencing (e.g. ABI SOLID, Illumina Genome Analyzer, Roche/454 GS FLX), flow cytometry (e.g. LUMINEX) and the like, that allow the analysis of differential miRNA expression levels between samples of a subject (e.g. diseased) and a control subject (e.g. healthy, reference sample). The sample material measure by the aforementioned means may be total RNA, labeled total RNA, amplified total RNA, cDNA, labeled cDNA, amplified cDNA, miRNA, labeled miRNA, amplified miRNA or any derivatives that may be generated from the aforementioned RNA/DNA species. By determining the miRNA expression profile, each miRNA is represented by a numerical value. The higher the value of an individual miRNA, the higher is the expression level of said miRNA, or the lower the value of an individual miRNA, the lower is the expression level of said miRNA.

The “miRNA expression profile”, as used herein, represents the expression level/expression data of a single miRNA or a collection of expression levels of at least two miRNAs, preferably of least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more, or up to all known miRNAs.

The term “differential expression” of miRNAs as used herein, means qualitative and/or quantitative differences in the temporal and/or local miRNA expression patterns, e.g. within and/or among biological samples, body fluid samples, cells, or within blood. Thus, a differentially expressed miRNA may qualitatively have its expression altered, including an activation or inactivation in, for example, blood from a diseases subject versus blood from a healthy subject. The difference in miRNA expression may also be quantitative, e.g. in that expression is modulated, i.e. either up-regulated, resulting in an increased amount of miRNA, or down-regulated, resulting in a decreased amount of miRNA. The degree to which miRNA expression differs need only be large enough to be quantified via standard expression characterization techniques, e.g. by quantitative hybridization (e.g. to a microarray, to beads), amplification (PCR, RT-PCR, qRT-PCR, high-throughput RT-PCR), ELISA for quantitation, next generation sequencing (e.g. ABI SOLID, Illumina Genome Analyzer, Roche 454 GS FL), flow cytometry (e.g. LUMINEX) and the like.

Nucleic acid hybridization may be performed using a microarray/biochip or in situ hybridization. In situ hybridization is preferred for the analysis of a single miRNA or a set comprising a low number of miRNAs (e.g. a set of at least 2 to 50 miRNAs such as a set of 2, 5, 10, 20, 30, or 40 miRNAs). The microarray/biochip, however, allows the analysis of a single miRNA as well as a complex set of miRNAs (e.g. a all known miRNAs or subsets therof).

For nucleic acid hybridization, for example, the polynucleotides (probes) according to the present invention with complementarity to the corresponding miRNAs to be detected are attached to a solid phase to generate a microarray/biochip (e.g. 37 polynucleotides (probes) which are complementary to the 37 miRNAs having SEQ ID NO: 1 to 37. Said microarray/biochip is then incubated with a biological sample containing miRNAs, isolated (e.g. extracted) from the blood sample from a subject such as a human or an animal, which may be labelled, e.g. fluorescently labelled, or unlabelled. Quantification of the expression level of the miRNAs may then be carried out e.g. by direct read out of a label or by additional manipulations, e.g. by use of a polymerase reaction (e.g. template directed primer extension, MPEA-Assay, RAKE-assay) or a ligation reaction to incorporate or add labels to the captured miRNAs. Alternatively, the polynucleotides which are at least partially complementary (e.g.a set of chimeric polynucleotides with each a first stretch being complementary to a set of miRNA sequences and a second stretch complementary to capture probes bound to a solid surface (e.g. beads, Luminex beads)) to miRNAs having SEQ ID NO: 1 to 37. are contacted with the biological sample containing miRNAs (e.g a body fluid sample, preferably a blood sample) in solution to hybridize. Afterwards, the hybridized duplexes are pulled down to the surface (e.g a plurality of beads) and successfully captured miRNAs are quantitatively determined (e.g. FlexmiR-assay, FlexmiR v2 detection assays from Luminex).

Nucleic acid amplification may be performed using real time polymerase chain reaction (RT-PCR) such as real time quantitative polymerase chain reaction (RT qPCR). The standard real time polymerase chain reaction (RT-PCR) is preferred for the analysis of a single miRNA or a set comprising a low number of miRNAs (e.g. a set of at least 2 to 50 miRNAs such as a set of 2, 5, 10, 20, 30, or 40 miRNAs), whereas high-throughput RT-PCR technologies (e.g. OpenArray from Applied Biosystems, SmartPCR from Wafergen, Biomark System from Fluidigm) are also able to measure large sets (e.g a set of 10, 20, 30, 50, 80, 100, 200 or more) to all known miRNAs in a high parallel fashion. RT-PCR is particularly suitable for detecting low abandoned miRNAs.

The aforesaid real time polymerase chain reaction (RT-PCR) may include the following steps: (i) extracting the total RNA from a blood cell sample derived from a blood sample of a subject, (ii) obtaining cDNA-transcripts by RNA reverse transcription (RT) reaction using universal or miRNA-specific RT primers (e.g. stem-loop RT primers); (iii) optionally amplifying the obtained cDNA-transcripts (e.g. by PCR such as a specific target amplification (STA)), (iv) detecting the miRNA(s) level in the sample by means of (real time) quantification of the cDNA of step (ii) or (iii) e.g. by real time polymerase chain reaction wherein a fluorescent dye (e.g. SYBR Green) or a fluorescent probe (e.g. Taqman probe) probe are added. In Step (i) the isolation and/or extraction of RNA may be omitted in cases where the RT-PCR is conducted directly from the miRNA-containing sample. Kits for determining a miRNA expression profile by real time polymerase chain reaction (RT-PCR) are e.g. from Life Technologies, Applied Biosystems, Ambion, Roche, Qiagen, Invitrogen, SABiosciences, Exiqon.

A variety of kits and protocols to determine an expression profile by real time polymerase chain reaction (RT-PCR) such as real time quantitative polymerase chain reaction (RT qPCR) are available. For example, reverse transcription of miRNAs may be performed using the TaqMan MicroRNA Reverse Transcription Kit (Applied Biosystems) according to manufacturer's recommendations. Briefly, miRNA may be combined with dNTPs, MultiScribe reverse transcriptase and the primer specific for the target miRNA. The resulting cDNA may be diluted and may be used for PCR reaction. The PCR may be performed according to the manufacturer's recommendation (Applied Biosystems). Briefly, cDNA may be combined with the TaqMan assay specific for the target miRNA and PCR reaction may be performed using ABI7300. Alternative kits are available from Ambion, Roche, Qiagen, Invitrogen, SABiosciences, Exiqon etc.

The term “subject”, as used in the context of the present invention, means a patient or individual or mammal suspected to be affected by a disease, preferably affected by Multiple Sclerosis (MS) and/or by Alzheimer's Disease (AD).

The term “control subject”, as used in the context of the present invention, may refer to a subject known to be affected with a disease, preferably AD and/or MS (positive control), i.e. diseased, or to a subject known to be not affected with a disease, preferably not affected by AD and/or MS (negative control), i.e. a healthy control subject. It may also refer to a subject known to be effected by another disease/condition. It should be noted that a control subject that is known to be healthy, i.e. not suffering from a disease, preferably not suffering from AD and/or MS, may possibly suffer from another disease not tested/known. The control subject may be any mammal, including both a human and another mammal, e.g. an animal such as a rabbit, mouse, rat, or monkey. Human “control subjects” are particularly preferred.

The inventors of the present invention surprisingly found that miRNAs are significantly dysregulated in blood samples of diseased subjects, preferably MS or AD subjects in comparison to a cohort of controls (healthy control subjects) and thus, miRNAs are appropriated biomarkers for diagnosing and/or prognosing of a disease, preferably are appropriated biomarkers for diagnosing and/or prognosing MS and/or AD in a non-invasive fashion or minimal-invasive fashion, preferably from a blood sample.

In a first aspect, the invention provides an isolated nucleic acid molecule comprising a nucleotide sequence presented as SEQ ID NO: 1-37, a fragment thereof, or a nucleotide sequence with at least 90%, 94%, 96% or greater sequence identity thereto.

The isolated nucleic acid molecules with SEQ ID NO: 1-37 are miRNA molecules (FIG. 1). Said miRNAs were found to be differentially expressed between healthy control (HC) and disease subjects (FIG. 3), such as Multiple Sclerosis (MS) (FIG. 4) or Alzheimer's Disease (AD) subjects (FIG. 5). Thus said novel miRNAs qualify to be employed as biomarkers in the diagnosis and/or prognosis of diseases, such as Multiple Sclerosis (MS) and/or Alzheimer's Disease (AD).

In a second embodiment of the first aspect of the invention, the invention provides an isolated nucleic acid molecule comprising a nucleotide sequence presented as SEQ ID NO: 38-69, a fragment thereof, or a nucleotide sequence with at least 90%, 94%, 96% or greater sequence identity thereto.

In a second aspect, the invention provides an isolated nucleic molecule that is a complement to nucleic acid molecules according to the first aspect of the invention (FIG. 6, 7).

In a third aspect, the invention provides a vector comprising isolated nucleic acid molecules according to the first aspect of the invention (FIG. 3).

Preferably, the vector comprises the isolated nucleic acid molecules according to the first aspect of the invention (with SEQ ID NO: 1-37), more preferably the vector comprises the isolated nucleic acid molecules with SEQ ID NO: 1 and/or SEQ ID NO: 4. It is understood that if said vector is a RNA-vector, it is the RNA-form of the isolated nucleotide molecule, its complement or a fragment thereof that is comprised in the vector. It is further understood that if said vector is a DNA-vector, it is the DNA-form of the isolated nucleotide molecule, its complement or a fragment thereof that is comprised in the vector.

In a further embodiment the vector is a pSG5 vector, comprising the DNA-form of the isolated nucleic acid molecules according to the first aspect of the invention (with SEQ ID NO: 1-37), more preferably, the vector is a pSG5 vector, comprising the isolated nucleic acid molecules with SEQ ID NO: 1 and/or SEQ ID NO:4.

In a fourth aspect, the invention provides a host cell that is transformed with the isolated nucleic acid molecules according to the first aspect of the invention (FIG. 9).

Preferably the host cell is transformed with the isolated nucleic acid molecules with SEQ ID NO: 1-37, more preferably the host cell is transformed with the isolated nucleic acid molecules with SEQ ID NO: 1 and/or SEQ ID NO:4. More preferably, the host cell is a human cell that is transformed with the isolated nucleic acid molecules with SEQ ID NO: 1-37, more preferably the host cell is a human cell transformed with the isolated nucleic acid molecules with SEQ ID NO: 1 and/or SEQ ID NO: 4. Even more preferably, the host cell is a human 293T cell transformed with the isolated nucleic acid molecules with SEQ ID NO: 1-37, more preferably the host cell is a human cell transformed with the isolated nucleic acid molecules with SEQ ID NO: 1 and/or SEQ ID NO: 4.

In a fifth aspect, the invention provides a host cell that is transformed with the vector according to the third aspect of the invention (FIG. 9).

Preferably, the host cell is transformed with the vector comprising the isolated nucleic acid molecules with SEQ ID NO: 1-37, more preferably the host cell is transformed with the vector comprising the isolated nucleic acid molecules with SEQ ID NO: 1 and/or SEQ ID NO: 4. More preferably the host cell is a human cell that is transformed with the vector comprising the isolated nucleic acid molecules with SEQ ID NO: 1-37, more preferably the host cell is human cell that is transformed with the vector comprising the isolated nucleic acid molecules with SEQ ID NO: 1 and/or SEQ ID NO: 4.

Even more preferably, the host cell is a human 293T cell that is transformed with the vector comprising the isolated nucleic acid molecules with SEQ ID NO: 1-37, more preferably the host cell is human cell that is transformed with the vector comprising the isolated nucleic acid molecules with SEQ ID NO: 1 and/or SEQ ID NO: 4.

In a further embodiment, host cell is a human 293T cell into which a pSG5-novel-miR-1005 expression plasmid, thus a vector comprising SED ID NO: 1 and 4, was transfected.

In a sixth aspect, the invention provides a primer for reverse transcribing an isolated nucleic acid molecule according to the first aspect of the invention (FIG. 7).

It is preferred to use either universal or specific primers for reverse transcribing the isolated nucleic acid molecules with SEQ ID NO: 1-37. It is preferred to use universal primers for reverse transcribing comprising a poly-T sequence motif. When using specific primer for reverse transcribing the isolated nucleic acid molecules with SEQ ID NO: 1-37, preferably said primers are partially complementary to the 3′-end of the isolated nucleic acid molecules with SEQ ID NO: 1-37. It is especially preferred to employ stem-loop RT primers for reverse transcribing the isolated nucleic acid molecules with SEQ ID NO: 1-37, prefereably for transcribing the isolated nucleic acid molecules with SEQ ID NO: 1, 2, 4.

In a seventh aspect, the invention provides a cDNA-transcript of an isolated nucleic acid molecule according to the first aspect of the invention (FIG. 7).

Said cDNA-transcript according to the fifth aspect of the invention is obtained from using the RT-primers according to the sixth aspect of the invention. Preferably, cDNA-transcript of miRNAs with SEQ ID NO: 1-37, more preferably cDNA-transcripts with SEQ ID NO: 1 or SEQ ID NO: 2 or SEQ ID NO: 4 are obtained when employing said RT-primers according to the sixth aspect of the invention.

In an eighth aspect, the invention provides a set of primer pairs for amplifying said cDNA-transcripts according to the seventh aspect of the invention (FIG. 7).

Preferably, primer pairs are provided for amplifying cDNA-transcripts of nucleic acid molecules with nucleotide sequence presented as SEQ ID NO: 1-37, more preferably primer pairs are for amplifying cDNA-transcripts of nucleic acid molecules with nucleotide sequence presented as SEQ ID NO: 1 or SEQ ID NO: 2.

In a ninth aspect, the invention provides polynucleotide for detecting an isolated nucleic acid molecule according to the first or second aspect of the invention. (FIG. 7, 9)

In a tenth aspect, the invention provides a cDNA-transcript according to the seventh aspect of the invention, hybridized to an isolated nucleic acid molecule according to the first aspect of the invention. Thus, said cDNA-transcripts (of the seventh aspect of the invention) form a duplex with the isolated nucleic acid molecule according to the first aspect of the invention (FIG. 7). Preferably, cDNA-transcripts derived from reverse transcribing miRNAs with SEQ ID NO: 1-37 are hybridized to miRNAs with SEQ ID NO: 1-37 are provided. It is hereby understood that said cDNA-miRNA duplexes according to the tenth aspect of the invention only include duplexes where the cDNA-transcript is derived from the identical miRNA it is hybridized to.

In an eleventh aspect, the invention provides an isolated nucleic acid molecules according to the first aspect of the invention for use in diagnosis and/or prognosis of a disease or the invention provides the use of an isolated nucleic acid molecules according to the first aspect of the invention for diagnosis and/or prognosis of a disease (FIG. 3).

A first embodiment of the eight aspect of the invention provides an isolated nucleic acid molecules according to the first aspect of the invention for use in diagnosis and/or prognosis of a disease. Herein, a isolated nucleic acid molecule, with the nucleotide sequence selected from group consisting of SEQ ID NO: 1-37 for use in diagnosis and/or prognosis of a disease is provided.

Preferably, the isolated nucleic acid molecules are for use in diagnosis and/or prognosis of Multiple Sclerosis (FIG. 4). It is preferred that isolated nucleic acid molecule selected from the group consisting of SEQ ID NO: 3, 5, 28, 23, 10, 30, 27, 35, 33, 19, 14, 21, 31, 37, 29, 7, 32, 24 or 22 are for use in diagnosis and/or prognosis of Multiple Sclerosis. It is further preferred that isolated nucleic acid molecule selected from the group consisting of:
(a) a nucleic acid molecule with a nucleotide sequence shown in SEQ ID NO: 3, 5, 28, 23, 10, 30, 27, 35, 33, 19, 14, 21, 31, 37, 29, 7, 32, 24 or 22
(b) a nucleic acid molecule with a nucleotide sequence which is the complement of the nucleotide sequence of (a),
(c) a nucleic acid molecule with a nucleotide sequence which comprises the nucleotide sequence of (a) or (b),
(d) a nucleic acid molecule with a nucleotide sequence of 16-21 nucleotides which is a fragment of the nucleotide sequence of (a) or (b) or (c),
(e) a nucleic acid molecule with a nucleotide sequence which has a sequence identity of at least 90%, 94%, 96% or greater to the nucleotide sequence set forth in (a) or (b) or (c) or (d)

for use in diagnosis and/or prognosis of Multiple Sclerosis are provided.

Preferably, the isolated nucleic acid molecules are for use in diagnosis and/or prognosis of Alzheimer's Disease (FIG. 5). It is preferred that isolated nucleic acid molecule selected from the group consisting of SEQ ID NO: 28, 14, 2, 11, 36, 24, 34, 22, 19, 12, 8, 13, 32, 26, 15, 10, 21, 18, 6, 17 or 2 are for use in diagnosis and/or prognosis of Alzheimer's Disease. It is further preferred that isolated nucleic acid molecule selected from the group consisting of:
(a) a nucleic acid molecule with a nucleotide sequence shown in SEQ ID NO: 28, 14, 2, 11, 36, 24, 34, 22, 19, 12, 8, 13, 32, 26, 15, 10, 21, 18, 6, 17 or 2
(b) a nucleic acid molecule with a nucleotide sequence which is the complement of the nucleotide sequence of (a),
(c) a nucleic acid molecule with a nucleotide sequence which comprises the nucleotide sequence of (a) or (b),
(d) a nucleic acid molecule with a nucleotide sequence of 16-21 nucleotides which is a fragment of the nucleotide sequence of (a) or (b) or (c),
(e) a nucleic acid molecule with a nucleotide sequence which has a sequence identity of at least 90%, 94%, 96% or greater to the nucleotide sequence set forth in (a) or (b) or (c) or (d)

for use in diagnosis and/or prognosis of Alzheimer's Disease are provided.

It is preferred that in the diagnosis and/or prognosis of a disease or of Multiple Sclerosis or of Alzheimer's Disease according to the first embodiment of the eleventh aspect of the invention said diagnosis and/or prognosis is from a blood sample, preferably from a whole blood sample, more preferably from the blood cell fraction isolated from a whole blood sample, most preferably from the blood cell fraction isolated from a whole blood sample comprising red blood cells, platelets and leukocytes or from the blood cell fraction isolated from a whole blood sample consisting of a mixture of red blood cells, platelets and leukocytes.

A second embodiment of the eleventh aspect of the invention provides the (in vitro) use of an isolated nucleic acid molecule according to the first aspect of the invention for diagnosis and/or prognosis of a disease. Herein, the (in vitro) use of isolated nucleic acid molecules, selected from group consisting of SEQ ID NO: 1-37 in diagnosis and/or prognosis of a disease is provided (FIG. 3).

Preferably, the (in vitro) use of isolated nucleic acid molecule in diagnosis and/or prognosis of Multiple Sclerosis is provided (FIG. 4). It is preferred that the (in vitro) use of isolated nucleic acid molecule selected from the group consisting of SEQ ID NO: 3, 5, 28, 23, 10, 30, 27, 35, 33, 19, 14, 21, 31, 37, 29, 7, 32, 24 or 22 in diagnosis and/or prognosis of Multiple Sclerosis is provided. Further the (in vitro) use of isolated nucleic acid molecule selected from the group consisting of:
(a) a nucleic acid molecule with a nucleotide sequence shown in SEQ ID NO: 3, 5, 28, 23, 10, 30, 27, 35, 33, 19, 14, 21, 31, 37, 29, 7, 32, 24 or 22
(b) a nucleic acid molecule with a nucleotide sequence which is the complement of the nucleotide sequence of (a),
(c) a nucleic acid molecule with a nucleotide sequence which comprises the nucleotide sequence of (a) or (b),
(d) a nucleic acid molecule with a nucleotide sequence of 16-21 nucleotides which is a fragment of the nucleotide sequence of (a) or (b) or (c),
(e) a nucleic acid molecule with a nucleotide sequence which has a sequence identity of at least 90%, 94%, 96% or greater to the nucleotide sequence set forth in (a) or (b) or (c) or (d)

in diagnosis and/or prognosis of Multiple Sclerosis is provided.

Preferably, the (in vitro) use of isolated nucleic acid molecule in diagnosis and/or prognosis of Alzheimer's Disease is provided (FIG. 5). It is preferred that the (in vitro) use of isolated nucleic acid molecule selected from the group consisting of SEQ ID NO: 28, 14, 2, 11, 36, 24, 34, 22, 19, 12, 8, 13, 32, 26, 15, 10, 21, 18, 6, 17 or 2 in diagnosis and/or prognosis of Alzheimer's Disease provided. Further the (in vitro) use of isolated nucleic acid molecule selected from the group consisting of:
(a) a nucleic acid molecule with a nucleotide sequence shown in SEQ ID NO: 28, 14, 2, 11, 36, 24, 34, 22, 19, 12, 8, 13, 32, 26, 15, 10, 21, 18, 6, 17 or 2
(b) a nucleic acid molecule with a nucleotide sequence which is the complement of the nucleotide sequence of (a),
(c) a nucleic acid molecule with a nucleotide sequence which comprises the nucleotide sequence of (a) or (b),
(d) a nucleic acid molecule with a nucleotide sequence of 16-21 nucleotides which is a fragment of the nucleotide sequence of (a) or (b) or (c),
(e) a nucleic acid molecule with a nucleotide sequence which has a sequence identity of at least 90%, 94%, 96% or greater to the nucleotide sequence set forth in (a) or (b) or (c) or (d)

in diagnosis and/or prognosis of Alzheimer's Disease is provided.

It is preferred that the (in vitro) use in diagnosis and/or prognosis of a disease or of Multiple Sclerosis or of Alzheimer's Disease according to the second embodiment of the eight aspect of the invention is from a blood sample, preferably from a whole blood sample, more preferably from the blood cell fraction isolated from a whole blood sample, most preferably from the blood cell fraction isolated from a whole blood sample comprising red blood cells, platelets and leukocytes or from the blood cell fraction isolated from a whole blood sample consisting of a mixture of red blood cells, platelets and leukocytes.

In a twelfth aspect, the invention provides an isolated nucleic acid molecules according to the first aspect of the invention for use as a medicament or the invention provides the (in vitro) use of an isolated nucleic acid molecules according to the first aspect of the invention for therapeutic intervention (therapy).

In a thirteenth aspect, the present invention provides a method for diagnosing and/or prognosing of a disease, comprising the steps:
(i) determining an expression profile of at least one isolated nucleic acid molecule as defined according to the first aspect of the invention, which is differentially expressed in a disease in a blood sample from a subject, and
(ii) comparing said expression profile to a reference, wherein the comparison of said expression profile to said reference allows for the diagnosis and/or prognosis of the disease,

Herein, it is preferred that the nucleotide sequence of said at least one nucleic acid molecule is selected from SEQ ID NO: 1-37, a fragment thereof, or a nucleotide sequence with at least 90%, 94%, 96% or greater sequence identity thereto (FIG. 3).

According to the present invention the expression profile is determined in a blood sample, preferably in a blood cell sample derived from a whole blood sample of a subject, preferably a human subject. Herein, the whole blood sample is collected from the subject by conventional blood draw techniques. Blood collection tubes suitable for collection of whole blood include EDTA- (e.g. K2-EDTA Monovette tube), Na-citrate-, ACD-, Heparin-, PAXgene Blood RNA-, Tempus Blood RNA-tubes. According to the present invention the collected whole blood sample, which intermediately may be stored before use, is processed to result in a blood cell sample of whole blood. This is achieved by separation of the blood cell fraction (the cellular fraction of whole blood) from the serum/plasma fraction (the extra-cellular fraction of whole blood). It is preferred, that the blood cell sample derived from the whole blood sample comprises red blood cells, white blood cells or platelets, it is more preferred that the blood cell sample derived from the whole blood sample comprises red blood cells, white blood cells and platelets, most preferably the blood cell sample derived from the whole blood sample consists of (a mixture of) red blood cells, white blood cells and platelets.

Preferably, the total RNA, including the miRNA fraction, or the miRNA-fraction is isolated from said blood cells present within said blood cell samples. Kits for isolation of total RNA including the miRNA fraction or kits for isolation of the miRNA-fraction are well known to those skilled in the art, e.g. miRNeasy-kit (Qiagen, Hilden, Germany), Paris-kit (Life Technologies, Weiterstadt, Germany). The miRNA-profile of said set comprising at least one nucleic acid molecule with nucleotide sequence selected from SEQ ID NO. 1 to 97 is then determined from the isolated RNA. The determination of the expression profile may be by any convenient means for determining miRNAs or miRNA profiles. A variety of techniques are well known to those skilled in the art, as defined above, e.g. nucleic acid hybridisation, nucleic acid amplification, sequencing, mass spectroscopy, flow cytometry based techniques or combinations thereof. Subsequent to the determination of an expression profile as defined above in step (i) of the method for diagnosing and/or prognosing of a disease, preferably AD and/or MS of the present invention, said method further comprises the step (ii) of comparing said expression profile (expression profile data) to a reference, wherein the comparison of said expression profile (expression profile data) to said reference allows for the diagnosis and/or prognosis of a disease, preferably said reference allows for the diagnosis and/or prognosis of AD and/or MS. The reference may be the reference (e.g. reference expression profile (data)) of a healthy condition (i.e. not a disease, preferably not a AD- or MS-condition), it may be the reference (e.g. reference expression profile (data)) of a diseased condition (i.e. a disease, preferably a disease such as AD and/or MS) or it may be the reference (e.g. reference expression profiles (data)) of at least two conditions from which at least one condition is a diseased condition (i.e. a disease, preferably a disease such as AD and/or MS). For example, (i) one condition may be a healthy condition (i.e. not a disease, preferably not AD or MS) and one condition may be a diseased condition (i.e. a disease, preferably AD and/or MS), or (ii) one condition may be a diseased condition (preferably AD and/or MS, or. a specific form of a said disease(s),) and one condition may be another diseased condition (preferably AD and/or MS, or. a another specific form of a said disease(s), or an other timepoint of treatement, other therapeutic treatment).

Further, the reference may be the reference expression profiles (data) of essentially the same, preferably the same, miRNAs (with nucleotide sequences presented as SEQ ID NO: 1-37) as in step (i), preferably in a blood sample originated from the same source (e.g. blood, blood cells as defined above) as the blood sample from the subject (e.g. human or animal) to be tested, but obtained from subjects (e.g. human or animal) known to not suffer from a disease, preferably AD and/or MS, and from subjects (e.g. human or animal) known to suffer from a disease (preferably AD and/or MS). It is understood that the reference expression profile is not necessarily obtained from a single subject known to be affected by a disease (preferably affected by AD and/or M)S or known to be not affected by the disease (e.g. healthy subject), but may be an average reference expression profile of a plurality of subjects known to be affected by a disease, or known to be not affected by a disease, e.g. at least 2 to 200 subjects, more preferably at least 10 to 150 subjects, and most preferably at least 20 to 100 subjects. The expression profile and the reference expression profile may be obtained from a subject/patient of the same species (e.g. human or animal), or may be obtained from a subject/patient of a different species (e.g. human or animal). Preferably, said expression profiles are obtained from the same species (e.g. human or animal), of the same gender (e.g. female or male) and/or of a similar age/phase of life (e.g. infant, young child, juvenile, adult) as the subject (e.g. human or animal) to be tested or diagnosed.

The comparison of the expression profile of the patient to be diagnosed (e.g. human or animal) to the (average) reference expression profile may then allow for diagnosing and/or prognosing of a disease, preferably AD and/or MS, or a specific form of said diseases.

In a particularly preferred embodiment of the method of the present invention, the reference is an algorithm or mathematical function. Preferably, the algorithm or mathematical function is obtained on the basis of the reference, preferably from thereference expression profiles (data) as defined above. It is preferred that the algorithm or mathematical function is obtained using a machine learning approach. Machine learning approaches may include but are not limited to supervised or unsupervised analysis: classification techniques (e.g. naïve Bayes, Linear Discriminant Analysis, Quadratic Discriminant Analysis Neural Nets, Tree based approaches, Support Vector Machines, Nearest Neighbour Approaches), Regression techniques (e.g. linear Regression, Multiple Regression, logistic regression, probit regression, ordinal logistic regression ordinal Probit-Regression, Poisson Regression, negative binomial Regression, multinomial logistic Regression, truncated regression), Clustering techniques (e.g. k-means clustering, hierarchical clustering, PCA), Adaptations, extensions, and combinations of the previously mentioned approaches.

According to the thirteenth aspect of the invention it is preferred that the the blood sample is preferably a whole blood sample, more preferably a blood cell fraction isolated from a whole blood sample, most preferably a blood cell fraction isolated from a whole blood sample comprising red blood cells, platelets and leukocytes or it is a blood cell fraction isolated from a whole blood sample consisting of a mixture of red blood cells, platelets and leukocytes.

Preferably, in the method according to the thirteenth aspect of the invention, the disease to be diagnosed and/or prognosed is selected from Multiple Sclerosis and/or Alzheimer's Disease (FIG. 4, 5).

More preferably, in the method according to the thirteenth aspect of the invention, the disease to be diagnosed and/or prognosed is Multiple Sclerosis. Thus, in the method for diagnosing and/or prognosing Multiple Sclerosis, the nucleotide sequence of the at least one isolated nucleic acid molecule is selected from the group consisting of SEQ ID NO: 3, 5, 28, 23, 10, 30, 27, 35, 33, 19, 14, 21, 31, 37, 29, 7, 32, 24 and 22, a fragment thereof, and a sequence having at least 90%, 94%, 96% or greater sequence identity thereto. (FIG. 4; miRNAs differentially expressed between Multiple Sclerosis and Healthy Control subjects)

More preferably, in the method according to the thirteenth aspect of the invention, the disease to be diagnosed and/or prognosed is Alzheimer's Disease. Thus, in the method for diagnosing and/or prognosing Alzheimer's Disease, the nucleotide sequence of the at least one isolated nucleic acid molecule is selected from the group consisting of SEQ ID NO: 28, 14, 2, 11, 36, 24, 34, 22, 19, 12, 8, 13, 32, 26, 15, 10, 21, 18, 6, 17 and 2, a fragment thereof, and a sequence having at least 90%, 94%, 96% or greater sequence identity thereto (FIG. 5; miRNAs differentially expressed between Alzheimer's Disease and Healthy Control subjects)

In a fourteenth aspect, the present invention provides means for determining the expression of at least one isolated nucleic acid molecule according to the first aspect of the invention, comprising
(a) one or more polynucleotides according to the ninth aspect of the invention, and
(b) a biochip, a RT-PCT system, a PCR-system, a flow cytometer, a Luminex system or a next generation sequencing system.

In a fifteenth aspect, the present invention provides a kit for diagnosing and/or prognosing a disease, comprising:
(a) means for determining the expression profile of at least one isolated nucleic acid molecule according to the first aspect of the invention
(b) one or more reference expression profiles

Herein the expression profile in (a) and the reference expression profiles in (b) are determined from at least one isolated nucleic acid molecule according to the first aspect of the invention in the same type of blood sample, preferably from age and sex-matched subjects.

In summary, the present invention is composed of the following items:
1. An isolated nucleic acid molecule comprising a nucleotide sequence presented as SEQ ID NO: 1-37, a fragment thereof, or a nucleotide sequence with at least 90%, 94%, 96% or greater sequence identity thereto.
2. An isolated nucleic molecule that is a complement to nucleic acid molecules of item 1.
3. A vector comprising an isolated nucleic acid molecule according to item 1 or 2.
4. A host cell transformed with an isolated nucleic acid molecule according item 1 or 2
5. A host cell transformed with the vector of item 3.
6. A primer for reverse transcribing an isolated nucleic acid molecule of item 1.
7. A cDNA-transcript of an isolated nucleic acid molecule of item 1.
8. A set of primer pairs for amplifying a cDNA-transcript of item 7.
9. A polynucleotide for detecting an isolated nucleic acid molecule of item 1 or 2.
10. A cDNA-transcript of item 7, hybridized to an isolated nucleic acid molecule of item 1.
11. An isolated nucleic acid molecule according to item 1, for use in diagnosing and/or prognosing of a disease.
12. An isolated nucleic acid molecule for use according to item 11, wherein the disease is Multiple Sclerosis and wherein the nucleic acid molecule is selected from the group consisting of:
   (a) a nucleotide sequence shown in SEQ ID NO: 3, 5, 28, 23, 10, 30, 27, 35, 33, 19, 14, 21, 31, 37, 29, 7, 32, 24 or 22
   (b) a nucleotide sequence which is the complement of the nucleotide sequence of (a),
   (c) a nucleotide sequence which comprises the nucleotide sequence of (a) or (b),
   (d) a nucleotide sequence of 16-21 nucleotides which is a fragment of the nucleotide sequence of (a) or (b) or (c),
   (e) a nucleotide sequence which has a sequence identity of at least 90%, 94%, 96% or greater to the nucleotide sequence set forth in (a) or (b) or (c) or (d)
13. An isolated nucleic acid molecule for use according to item 11, wherein the disease is Alzheimer's Disease and wherein the nucleic acid molecule is selected from the group consisting of:
   (a) a nucleotide sequence shown in SEQ ID NO: 28, 14, 2, 11, 36, 24, 34, 22, 19, 12, 8, 13, 32, 26, 15, 10, 21, 18, 6, 17 or 2
   (b) a nucleotide sequence which is the complement of the nucleotide sequence of (a),
   (c) a nucleotide sequence which comprises the nucleotide sequence of (a) or (b),
   (d) a nucleotide sequence of 16-21 nucleotides which is a fragment of the nucleotide sequence of (a) or (b) or (c),
   (e) a nucleotide sequence which has a sequence identity of at least 90%, 94%, 96% or greater to the nucleotide sequence set forth in (a) or (b) or (c) or (d)
14. The isolated nucleic acid for use according to any of the items 11 to 13, wherein the diagnosing and/or prognosing is from a blood sample, preferably from a whole blood sample, more preferably from the blood cell fraction isolated from a whole blood sample, most preferably from the blood cell fraction isolated from a whole blood sample comprising red blood cells, platelets and leukocytes or from the blood cell fraction isolated from a whole blood sample consisting of a mixture of red blood cells, platelets and leukocytes.
15. An isolated nucleic acid molecule according to item 1, for use as a medicament.
16. A method for diagnosing and/or prognosing of a disease comprising the steps of:
   (i) determining an expression profile of a set comprising at least one nucleic acid molecule as defined in item 1, which is differentially expressed in the disease in a blood sample from a subject, and
   (ii) comparing said expression profile to a reference, wherein the comparison of said expression profile to said reference allows for the diagnosis and/or prognosis of the disease
   wherein the nucleotide sequence of said at least one nucleic acid molecule is selected from SEQ ID NO: 1-37, a fragment thereof, or a nucleotide sequence with at least 90%, 94%, 96% or greater sequence identity thereto.
17. The method according to item 16, wherein the blood sample is preferably a whole blood sample, more preferably a blood cell fraction isolated from a whole blood sample, most preferably a blood cell fraction isolated from a whole blood sample comprising red blood cells, platelets and leukocytes or it is a blood cell fraction isolated from a whole blood sample consisting of a mixture of red blood cells, platelets and leukocytes.
18. The method according to any of the items 16 or 17, wherein the disease is selected from Multiple Sclerosis or Alzheimer's Disease
19. The method according to any of the items 16 to 18, wherein the disease is Multiple Sclerosis and wherein the nucleotide sequence of the at least one isolated nucleic acid molecule is selected from the group consisting of SEQ ID NO: 3, 5, 28, 23, 10, 30, 27, 35, 33, 19, 14, 21, 31, 37, 29, 7, 32, 24 and 22, a fragment thereof, and a sequence having at least 90%, 94%, 96% or greater sequence identity thereto.
20. The method according to any of the items 16 to 18, wherein the disease is Alzheimer's Disease and wherein the nucleotide sequence of the at least one isolated nucleic acid molecule is selected from the group consisting of SEQ ID NO: 28, 14, 2, 11, 36, 24, 34, 22, 19, 12, 8, 13, 32, 26, 15, 10, 21, 18, 6, 17 and 2, a fragment thereof, and a sequence having at least 90%, 94%, 96% or greater sequence identity thereto.
21. Means for determining the expression profile of at least one isolated nucleic acid molecule, comprising:
   (a) one or more polynucleotides according to item 9, and
   (b) a biochip, a RT-PCT system, a PCR-system, a flow cytometer, a Luminex system or a next generation sequencing system
   wherein the nucleotide sequence of said at least one nucleic acid molecule is selected from SEQ ID NO: 1-37, a fragment thereof, or a nucleotide sequence with at least 90%, 94%, 96% or greater sequence identity thereto.
22. The means according to item 21, wherein the one or more polynucleotides comprise
   a. a primer for reverse transcribing at least one isolated nucleic acid molecule according to item 6 and
   b. a set of primer pairs for amplifying at least one cDNA-transcript according to item 8
23. A kit for diagnosing and/or prognosing a disease, comprising:
   (a) means for determining the expression profile of at least one isolated nucleic acid molecule according to any of the items 21 or 22
   (b) one or more reference expression profiles
   wherein the expression profile in (a) and the reference expression profiles in (b) are determined from the same at least one nucleic acid molecule as defined in item 1 in the same type of blood sample, preferably from age and sex-matched subjects.
24. The kit according to item 23, wherein said kit is for diagnosing and/or prognosing Multiple Sclerosis and wherein the nucleotide sequence of the at least one isolated nucleic acid molecule is selected from the group consisting of SEQ ID NO: 3, 5, 28, 23, 10, 30, 27, 35, 33, 19, 14, 21, 31, 37, 29, 7, 32, 24 and 22, a fragment thereof, and a sequence having at least 90%, 94%, 96% or greater sequence identity thereto.
25. The kit according to item 23, wherein said kit is for diagnosing and/or prognosing Alzheimer's Disease and wherein the nucleotide sequence of the at least one isolated nucleic acid molecule is selected from the group consisting of SEQ ID NO: 28, 14, 2, 11, 36, 24, 34, 22, 19, 12, 8, 13, 32, 26, 15, 10, 21, 18, 6, 17 and 2, a fragment thereof, and a sequence having at least 90%, 94%, 96% or greater sequence identity thereto.

### BRIEF DESCRIPTION OF THE DRAWINGS

FIG. 1: Novel isolated nucleic acid molecules (miRNAs) with SEQ ID NO: 1 to 37. The table shows the novel mature miRNA molecules identified by the present invention. With SEQ ID NO: =sequence identification number; miRNA=identifier of the novel isolated nucleic molecules assigned by the inventors; sequence miRNA=sequence of the novel isolated nucleic (miRNA) molecules in 5 ‘-3’-direction.

FIG. 2: Novel isolated nucleic acid molecules (miRNA precursors) with SEQ ID NO: 38 to 69. With SEQ ID NO: =sequence identification number; precursor=identifier of the novel isolated nucleic molecules assigned by the inventors; sequence precursor=sequence of the novel isolated nucleic (miRNA precursor) molecules in 5′-3′-direction.

FIG. 3: Isolated nucleic acid molecules (miRNAs) for diagnosing and/or prognosing a disease (miRNAs differentially expressed between diseased subjects and Healthy Control subjects). With SEQ ID NO: =sequence identification number; NGS reads, absolute=overall reads obtained by next generation sequencing analysis; normalized reads control=normalized NGS reads obtained in healthy control (HC) subjects; normalized reads AD=normalized NGS reads obtained in Alzheimer Disease (AD) subjects; normalized reads MS=normalized NGS reads obtained in Multiple Sclerosis (MS) subjects; max fold change=maximum fold change of differential expression of the respective novel miRNA obtained when comparing either AD or MS subjects with HC subjects, therefore allowing for use of said miRNAs in the diagnosis and/or prognosis of diseases. Depicted are only miRNA for which differential expression with a fold change of at least 2 (2-fold up- or down-regulation) was observed in said comparisons, namely AD versus HC or MS versus HC.

FIG. 4: Isolated nucleic acid molecules (miRNAs) for diagnosing and/or prognosing Multiple Sclerosis (miRNAs differentially expressed between Multiple Sclerosis and Healthy Control subjects). With SEQ ID NO: =sequence identification number; NGS reads, absolute=overall reads obtained by next generation sequencing analysis; normalized reads control=normalized NGS reads obtained in healthy control (HC) subjects; normalized reads MS=normalized NGS reads obtained in Multiple Sclerosis (MS)subjects; max fold change=maximum fold change of differential expression of the respective novel miRNA obtained when comparing MS with HC subjects, therefore allowing for use of said miRNAs in the diagnosis and/or prognosis of MS. Depicted are only miRNA for which differential expression with a fold change of at least 2 (2-fold up- or down-regulation) was observed.

FIG. 5: Isolated nucleic acid molecules (miRNAs) for diagnosing and/or prognosing Alzheimer's Disease (miRNAs differentially expressed between Alzheimer's Disease and Healthy Control subjects). With SEQ ID NO: =sequence identification number; NGS reads, absolute=overall reads obtained by next generation sequencing analysis; normalized reads control=normalized NGS reads obtained in healthy control (HC) subjects; normalized reads AD=normalized NGS reads obtained in Alzheimer Disease (AD) subjects; max fold change=maximum fold change of differential expression of the respective novel miRNA obtained when comparing AD with HC subjects, therefore allowing for use of said miRNAs in the diagnosis and/or prognosis of AD. Depicted are only miRNA for which differential expression with a fold change of at least 2 (2-fold up- or down-regulation) was observed.

FIG. 6: novel isolated nucleic acid molecules: novel-miR-1005 (miRNA precursor) and the 2 miRNAs derived thereof, novel-miR-1005 (SEQ ID NO: 1) and novel-miR-1005* (SEQ ID NO: 4). The secondary structures of precursor novel-mir-1005 depicted correlates well with the expectation of a typical miRNA-precursor and its mayor and minor miRNAs.

FIG. 7: Validation of the novel isolated nucleic acid molecules (miRNAs) with SEQ ID NO: 1, 2 and 4 by qRT-PCR. Shown are the amplification products of qRT-PCR on Bioanalyzer DNA 1000 Chip. As the used qRT-PCR system depends on poly-adenylation at the 3′ end of mature miRNAs followed by reverse transcription using an oligo-dT RT-primer that includes a universal tag sequence for the qPCR, amplification products of mature miRNAs are approximately 80-95 bps depending on the number of Adenine units added to the miRNA sequence during reverse transcription. The ladder bands shown on the left represent 50 and 100 bps. For the 3 miRNAs with SEQ ID NO: 2, 4, and 1 specific bands at 80-90 bps could be detected. With Pool 1, 2, 3=Agilent Bioanalyzer runs performed after qRT-PCR in 3 pools of 200 ng total RNA each, isolated from 15 AD and MS patients (each pool representing a mixture of AD and MS subjects); NTC=run of no template PCR control for each specific primer; RT-=run of RT reaction without enzyme; NTRT=run no template control for reverse transcription.

FIG. 8: Histogram blot of the absolute value of average z-scores from early versions of miRBase. With increasing version the distance from the initial miRNAs increases significantly. The overlap between the initial miRNAs up to version 7 and the novel miRNAs are presented on the right hand side of the plot.

FIG. 9: Validation of the novel isolated nucleic acid molecules (miRNAs) with SEQ ID NO: 1 and 2 by cloning into vector pSG5, transfection into host cell HEK293T, subsequent expression and detection by Northern analysis. Shown are the Northern blost detecting mature miRNAs novel-miR-1005-5p (novel-miR-1005*; SEQ ID NO: 4) and novel-miR-1005-3p (novel-miR-1005, SEQ ID NO: 1) with sequence specific radio-labeled probes in HEK293T cells transfected with pSG5 vector with inserted novel-mir-1005 precursor sequence (SEQ ID NO: 38). Loading control demonstrates equal RNA amounts in all lanes.

FIG. 10 A-D: distribution of features across miRBase versions. For different features, the distribution across different miRBase versions is presented as Box-Whisker plots. Also the novel miRNAs discovered in our study (depicted as miRBase version 100) are included as the right-most Box-Whisker plot.

### EXAMPLES

The Examples are designed in order to further illustrate the present invention and serve a better understanding. They are not to be construed as limiting the scope of the invention in any way.

Patient Samples:

Local ethics committees approved the study and patients gave written informed consent. All samples in this study have been evaluated in a blinded manner.

2.5 ml of whole blood of healthy controls (HC), Alzheimer's Disease (AD) subjects (n=15) and Multiple Sclerosis (MS) subjects (n=15) were drawn into PAXgene Blood RNA tubes (PreAnalytix GmbH, Hombrechtikon). The total RNA input required for NGS library preparation was obtained as follows: the blood cells preparation was derived from processing the whole blood samples by centrifugation. Herein, the whole blood collected in PAXgene Blood RNA tubes was spun down by a 10 min, 5000×g centrifugation. The blood cell pellet (the cellular blood fraction comprising red blood cells, white blood cells and platelets) formed at the bottom of the tube upon centrifugation was harvested for further processing, while the supernatant (including the extra-cellular blood fraction) was discarded. Total RNA, including the small RNA (miRNA-fraction) was extracted from the harvested blood cells (blood cell pellet) using the PAXgene Blood miRNA Kit (Qiagen GmbH, Hilden, Germany) according to the manufacturers protocol. The total RNA (including the microRNA) obtained was quantified using the NanoDrop 1000 and stored at −20° C. before use in the downstream experiments. For quality control of the total RNA, 1 μl of total RNA was applied on Agilent's Bioanalyzer, selecting either Agilent's nano- or pico-RNA Chip depending on RNA concentration determined by NanoDrop measurement.

Library Preparation & Next Generation Sequencing (NGS)

For the library preparation, the eluates from the RNA isolation were used. Library preparation was performed following the protocol of the TruSeq Small RNA Sample Prep Kit (Illumina, San Diego, US). To reduce adapter dimerization, only used half the amount of adapters was used during the preparation. Concentration of the ready prepped NGS-libraries was measured on the Agilent Bioanalyzer using the High Sensitivity Chip. The NGS libraries of the individual HC, MS and AD samples were then subjected at a concentration of 18 pmol for each lane of a flowcell using the cBot (Illumina). Sequencing of 50 cycles was performed on a HiSeq 2000 (Illumina, San Diego, US). Demultiplexing of the raw sequencing data and generation of the fastq files was done using CASAVA v.1.8.2.

Novel miRNA Sequence Features

From each miRNA precursor sequence and the two mature miRNAs, we calculated the following 24 features: the minimum free energy of the precursor, the 3p- and the 5p-miRNA using RNAfold (3 features), the percentage of bases A, C, U, G in the precursor, 3p- and 5p-miRNA (12 features), the precursor length, length of 3p and 5p mature forms (3 features), the loop length (1 feature), the distance to the next precursor in the genome in base pairs (computed from the genomic start positions of the precursors), and the number of precursors within windows of different genomic ranges (5 kb, 10 kb, 50 kb and 106 kb; 5 features). The windows were computed symmetrically around the middle of a precursor, and we counted also precursors that did not lie completely in the window, but overlapped with it. Since the miRBase provides the stem-loop sequences, we trimmed these sequences to obtain precursor sequences that start and end with the 5p/3p miRNAs, respectively.

Prediction of Novel miRNAs:

To predict novel miRNAs from the NGS sequencing reads we applied the miRDeep algorithm as integrated in the miRDeep2 pipeline using the default program parameters. We ran the miRDeep prediction algorithm on each sample separately. After the prediction, we extracted first for each sample the predicted novel miRNAs that had a signal-to-noise ratio of >=10 according to miRDeep. In order to avoid multiple miRNA predictions from different samples that are just shifted by few bases, we merged overlapping precursors. In detail, we extracted all miRNAs on the same chromosome that had overlapping genomic positions. If both miRNAs of a precursor shared an overlap of at least 11 bases, we took one of the overlapping precursors as representative for the novel predicted precursors at this location.

Matching to known RNA resources: As first step to exclude potential false positive miRNAs, we mapped the proposed novel miRNAs from the miRDeep algorithm back to other human non-coding RNA resources using BLAST (v 2.2.24). The set of databases contains miRBase v21, snoRNA-LBME-db, ncRNAs from Ensembl ‘Homo_sapiens.GRCh37.67.ncrna.fa’, and NONCODE (v3.0). We excluded sequences that aligned with >90% of their length (allowing 1 mismatch) to any of the above non-coding RNA sequences.

Biostatistical analysis: To estimate whether a specific miRBase version or set of miRBase versions deviates in one of the 24 features significantly from others, we carried out analysis of variance for each feature separately. All findings with FDR corrected significance values below 0.05 were considered significant. Since the considered features are on different scales, we applied for each feature a transformation to unit variance and centered them to zero, corresponding to z-scores. The standardized data have then been used for multivariate analysis including clustering or Principal Component Analysis (PCA). To cluster the miRBase versions, we applied complete linkage hierarchical clustering on the 24 scaled features. To limit the influence of single features we additionally cut the z-scores at an absolute threshold of 3. The PCA was carried out to produce a low dimensional representation of the miRBase versions. To calculate a distance of a miRNA precursor from a set of precursors, we first calculated the mean and standard deviation of each feature for the set of miRNAs. Then, we computed the z-scores for all features and the precursor, showing how many standard deviations this precursor is above or below the mean of the precursor set. To reduce the influence of single features, again absolute z-score values have been cut at 3. For all features, the average absolute value of the z-score has been calculated. Finally, we computed the absolute distance of the average z-score from the mean of the reference distribution as the final score to indicate how similar or different a precursor is to the reference distribution of precursors. All statistical calculations have been carried out in the freely available statistical programming environment R (version 3.0.2).

Validation of Novel miRNAs with qRT-PCR

To validate expression of novel miRNAs in blood samples, we selected novel miRNAs and performed quantitative real-time PCR. In detail, we pooled total RNA isolated from PAXgene blood tubes of 15 patients with Alzheimer's disease and 15 patients with Multiple Sclerosis into three RNA pools. Of each pool, 200 ng total RNA was reverse transcribed in 10 pl total volume containing 2 pl HighSpec buffer, 1 pl Nucleic Mix and 1 pl RT (components of miScript II RT kit, Qiagen, Hilden, Germany). Real-time PCR was conducted in 20 pl total volume using 1 pl of 1:10 diluted RT reaction, 10 pl QuantiTect SYBR Green Master Mix, 2 pl Universal Primer, 2 pl specific Primer Assay and 5 pl RNase-free water (Qiagen, Hilden, Germany). Negative controls included a no template controls for reverse transcription (NTRT), a RT reaction without enzyme (RT-) and a no template PCR control for each specific primer (NTC). All reactions were set up in duplicates. Specific amplification of novel miRNAs was satisfactorily demonstrated by a qRT-PCR product with a) a melting temperature of 75° C.+−1.5 C°; b) a mean raw Ct value of the product in the three pools of <35 and c) an assay dependent product length of 80-90 bp as evidenced on an DNA 1000 Bioanalyzer chip (Agilent Technologies).

Validation of Novel miRNAs with Cloning, Cell Lines and Northern Blots

(a) Cloning

For cloning of the pSG5-novel-miR-1005 expression plasmid, nucleotides 100841490-100841859 from Chromosome 11 were amplified from genomic DNA using specific primers (Forward:5′ GTAGTCCTGAAACGAGGGAG3′;Reverse:5′ GAGAGTCTGT GGCTTTTGA GG3′) by PCR and ligated via BglII and BamHI restriction sites into the pSG5 vector (Stratagene, La Jolla, USA).

(b) Cell Lines, Tissue Culture and Transfection

Human 293T cells were purchased from the German Collection of Microorganisms and Cell Cultures (DSMZ, Braunschweig, Germany). The transfection of 293T cells was carried out according to the manufacture's protocol using PolyFect transfection reagent (Qiagen, Hilden, Germany).

(c) Northern Blotting

The total RNA from pSG5 or pSG5-novel-miR-1005 transfected 293T cells respectively was isolated using QIAzol lysis reagent (Qiagen, Hilden, Germany) according to the manufacture's manual. Northern blotting was performed as described previously (23). The novel-miRNAs—novel-miR.1005-5p and novel-miR-1005-3p were detected with the following radioactive polynucletiodes (probes:)
AGAGGCTGAATTCCCAGTGAGTCCTGTCTC (for detecting novel-miR-1005-5p, SEQ ID NO: 4),ATTCGCTGGGAATTCAGCCTCTCCTGTCTC (for detecting novel-miR-1005-3p, SEQ ID NO: 1).

Results and Discussion

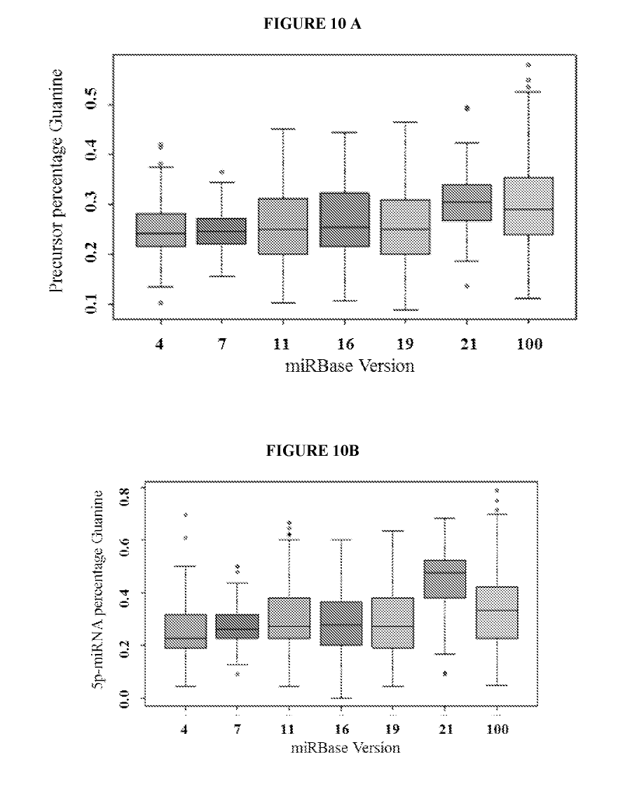

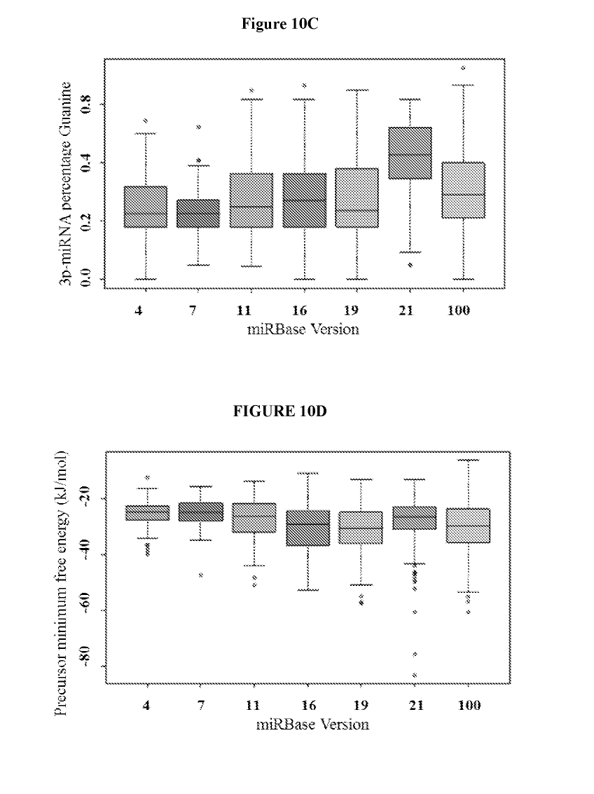

We defined a set of 24 sequence and structural features for all known miRNAs from miRBase version 1 to 21. These contain the minimum free energy, base composition, miRNA length and many others. Since the set of features partially considers the 3p and 5p miRNAs stemming from one precursor, we only included precursors with two annotated forms in our analysis. Each precursor has also been assigned with the first miRBase version its accession number has been mentioned in the miRBase, which means that each precursor is only taken into account for the miRBase version it was first listed and not for later versions. Since the first versions of the miRBase contain predominantly the stem loop sequences, i.e. the product of the processed pri-miRNA by DICER, and the later versions the actual precursor sequences that are trimmed at the 5′ and 3′ end of the two mature miRNAs, we would potentially observe a bias towards shorter precursor sequences with increasing miRBase versions. To account for this effect, we performed all analyses on the actual precursor sequences and trimmed all miRBase sequences accordingly. First, we considered changes of the features for each miRBase version separately. Since in some cases, however, just few novel precursors have been added, we grouped the versions in 6 batches: (1) version 1-4, (2) version 5-7, (3) version 8-11, (4) version 12-16, (5) version 17¬19 and (6) version 20-21. ANOVA testing suggested that all of the 24 features significantly vary dependent on the miRBase versions (FDR adjusted p-value below 0.05. Considering the base composition, we noticed an increase of Guanine (G) (FIG. 10A) and Cytosine (C) and a decrease of Adenine (A) and Uracil (U) in the precursor sequence from the first miRBase versions (1-4) of 24%, 21%, 25%, and 30% to 30%, 30%, 17%, and 23% in the last miRBase versions (20-21), respectively. Correspondingly, we observed this increase in percentage of Guanine for the 5p-miRNA (23% to 48%) and for Cytosine in the 3p-miRNA (23% to 43%) (FIGS. 10B and 10C), as well as a decrease of Uracil in the 5p-miRNA (32% to 17%) and of Adenine in the 3p-miRNA (24% to 13%). Despite this shift to a higher G/C content, which should have a stabilizing effect, the minimum free energy for the precursor is only slightly lower when comparing versions 1-4 and 20-21 (FIG. 10D). The minimum free energy of the precursor is an important feature that may directly influence the secondary structure. The minimum free energy increased from version 1-4 (−24.7 kj/mol), 5-7 (−24.8 kj/mol), 8-11 (¬26.1 kj/mol), 12-16 (−29 kj/mol) to −30.45 kj/mol in versions 17-19 and decreased again to −26.35 kj/mol in versions 20-21. Besides these sequence and structural features, we also observed some differences in the chromosomal clustering of precursors. While in versions 5-7 we found a maximum median value of 9.5 precursors in a 50 kb window around the precursors in the set, we detected a median value of 0 for the remaining later versions (adjusted p-value of 8.5*10-69). Using the 24 described features, we propose a method for prioritizing predicted precursors/miRNAs to facilitate further experimental validation.

In several case-control studies, we carried out next generation sequencing from blood of altogether 705 individuals. For each individual a separate sequencing library preparation followed by sequencing on Illumina HiSeq has been carried out. Altogether, we generated a total of 9.7 billion miRNA reads for the 705 samples (approximately 13.5 Million reads per sample). By applying miRDeep2, we generated a set of 1,452 potentially novel miRNA precursors. After mapping them back in a first step to different RNA resources as described in the Methods section, aiming to exclude initial false positive candidates, still 518 miRNA precursor candidates remained. For these, we calculated the same features as described above and included them also in the Box-Whiskers in FIG. 10A to D (grey boxes at the right edge). As these data show, the novel miRNAs match well to the later miRBase versions 17-19 or 20-21 while only a small portion seems to be close to the miRNAs in early versions (1-4, 5-7) of the miRBase. The latter may be the most promising novel miRNAs, minimizing a potential NGS bias. To show the similarity of miRNAs to each other with respect to the 24 features, we carried out a hierarchical clustering.

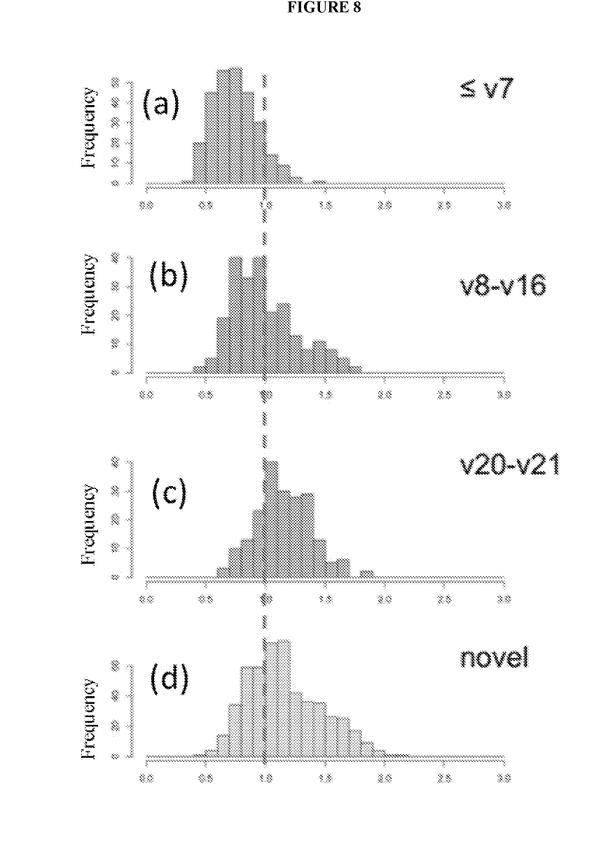

A key challenge for differentiating between true and false positive miRNA candidates is the availability of a reasonable positive set (i.e. actually validated miRNAs) and negative set (i.e. sequences that are no miRNAs). While at least the early miRBase versions represent such a positive set, all negative sets may show inherent bias. We thus implemented an approach, which relies just on the distance from the core miRNAs and extracted those miRNAs that matched the early versions best in the overall feature pattern. As reference we considered the early miRBase versions (1-7) and calculated for each of the features the z-score. To minimize the influence of single features, the maximal absolute z-score was set to 3. The mean value of the absolute z-scores was then calculated, representing the distance of the miRNAs from an “average” miRNA. Based on the mean and standard deviation in version 1-7, we also calculated distances for the remaining miRBase versions and the novel miRNAs. These are shown as histogram plots in FIG. 8. Here, the middle versions (v8-v16) show still a good proximity to the early versions while the later versions 20-21 and especially the novel miRNAs from our study are shifted significantly to the right, corresponding to higher distances from the reference distribution. For each of the novel miRNAs, we computed now their absolute distance from the mean of the reference distribution (averaged z-scores of features for miRNAs from miRBase versions 1-7) and sorted the resulting list in ascending order. The smaller the distance, the more similar should the miRNA be compared to the reference distribution.

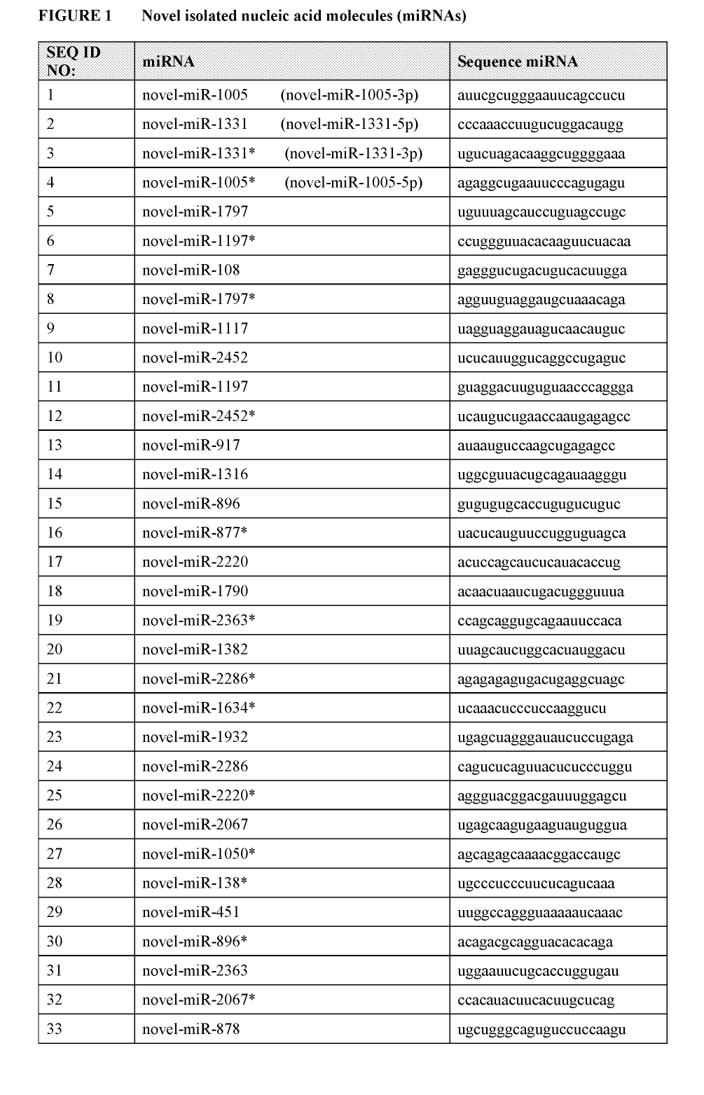

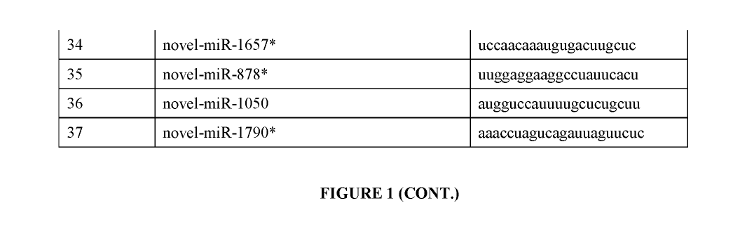

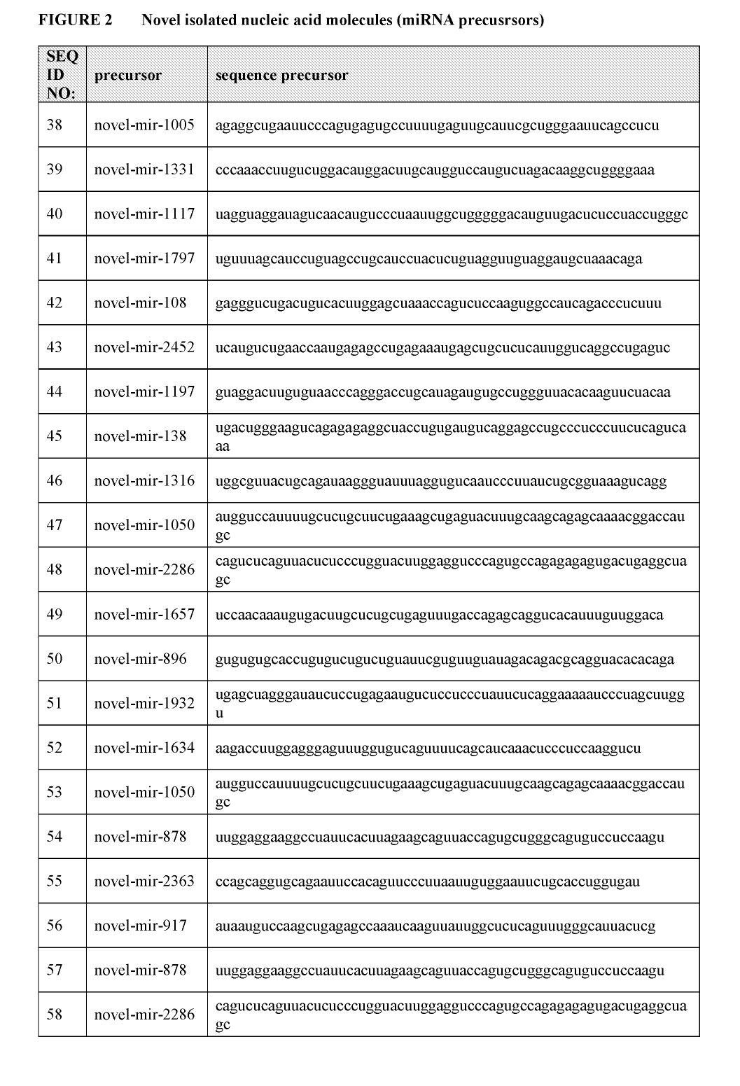

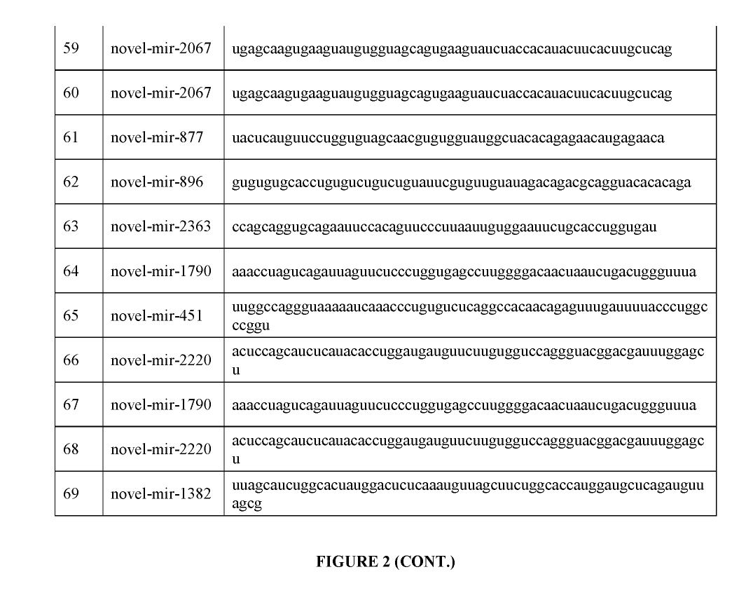

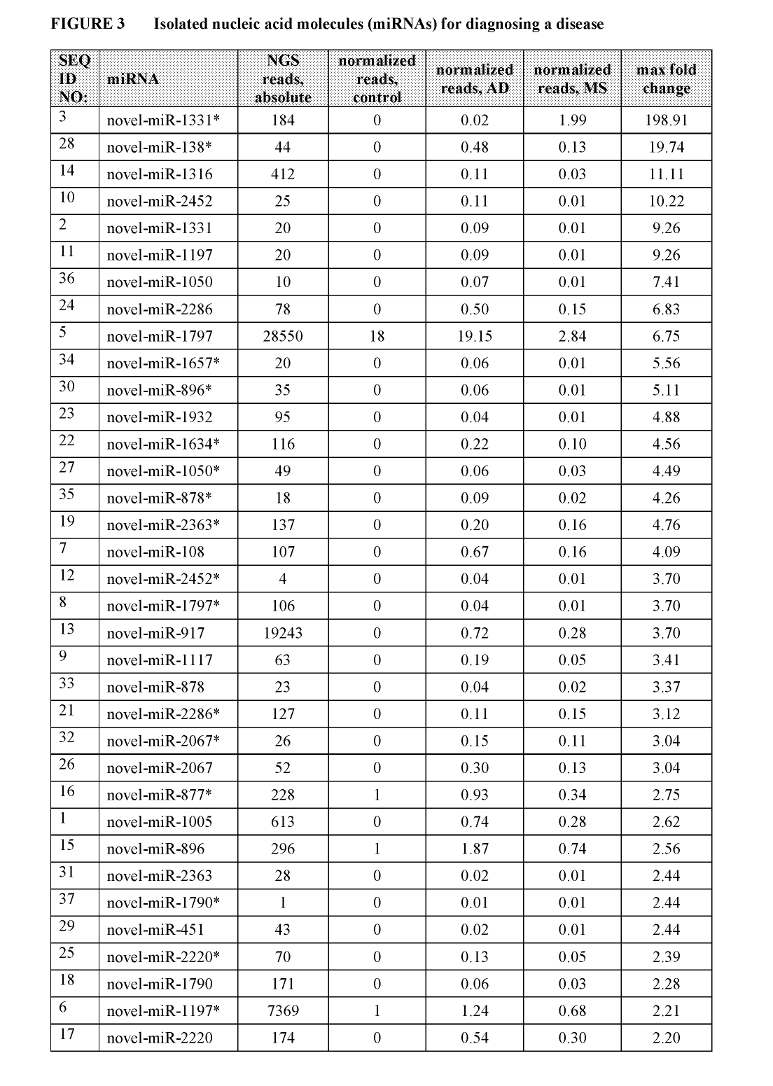

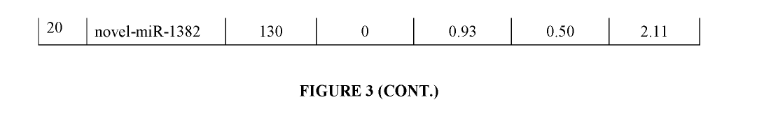

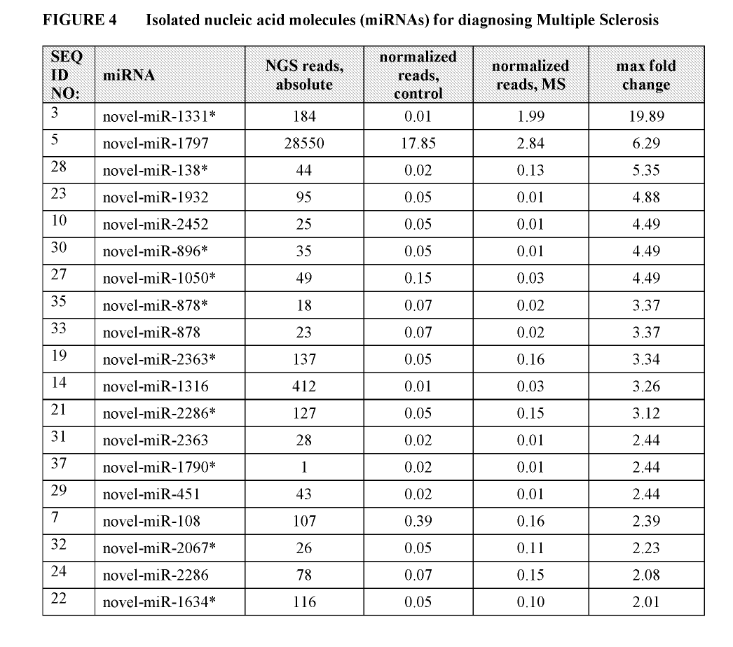

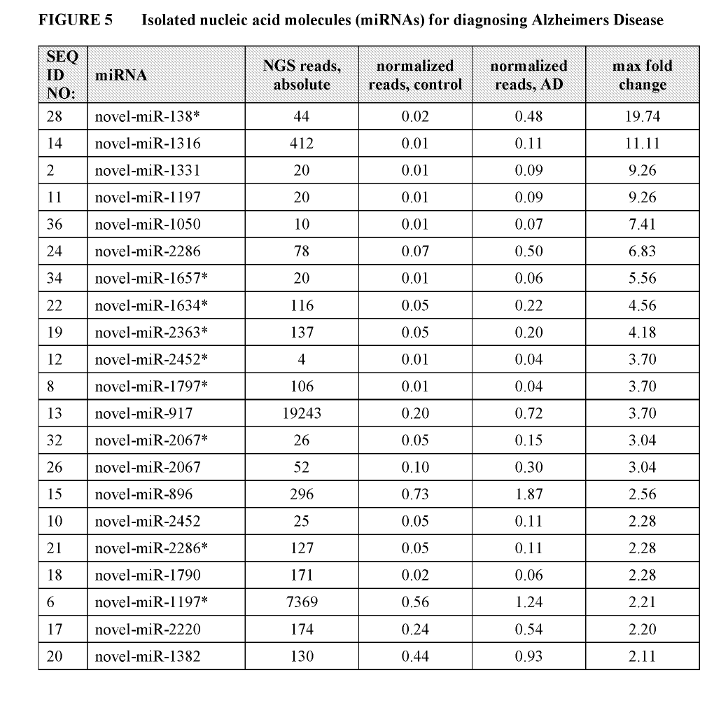

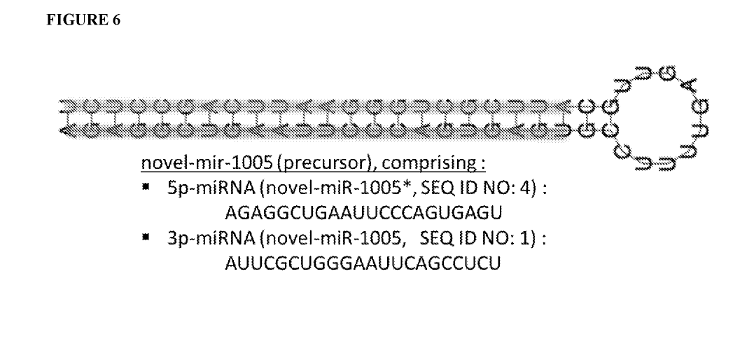

Following the described procedure, were were able to identify 37 novel mature miRNAs (FIG. 1, SEQ ID NO: 1-37) which are derived from the corresponding novel miRNA-precursors (FIG. 2, SEQ ID NO: 38-69).

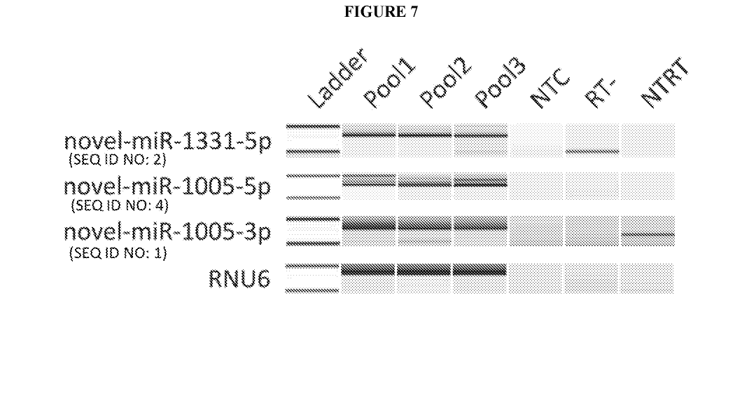

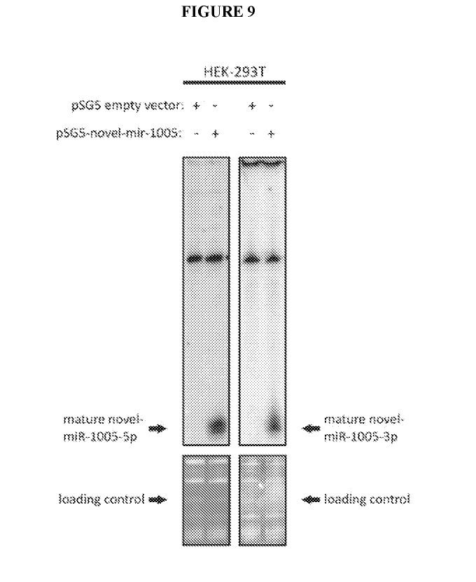

For experimental validation, we picked mature miRNAs and performed quantitative real-time PCR. Specific amplification products were obtained for the novel mature miRNAs (FIG. 7). As validation of correct processing of a novel precursor, we expressed novel-mir-1005 in HEK293T cells and performed northern blots to confirm presence of mature novel-miR-1005-5p (SEQ ID NO: 4) and novel-miR-1005-3p (SEQ ID NO: 1). As seen in FIG. 9, novel-mir-1005 precursor (SEQ ID NO: 38) has been processed into both mature forms, demonstrating its functional processing in the DICER complex.

### CONCLUSION

Our analysis of miRNA properties between different miRBase versions shows a substantial influence of all considered features depending of the version of this reference database. Generally, we observe a tendency of decreasing similarity from the initial miRBase versions for almost all considered features. Especially the increasing usage of complex high-throughput approaches along with respective in silico methods makes a certain percentage of false positive miRNAs likely. While these results do not imply that even the miRNAs with very aberrant features are actually no miRNAs but false positives, we assume that the likelihood of true miRNAs among those with similar features are higher.

1. An isolated nucleic acid molecule comprising a nucleotide sequence selected from the group consisting of SEQ ID NO: 2, SEQ ID NO: 1, SEQ ID NO: 3 to SEQ ID NO: 37, a fragment thereof, and a nucleotide sequence with at least 90%, 94%, 96% or greater sequence identity thereto.

2. An isolated nucleic molecule that is a complement to nucleic acid molecules of claim 1.

3. A vector comprising an isolated nucleic acid molecule according to claim 1.

4. A host cell transformed with an isolated nucleic acid molecule according claim 1.

5. (canceled)

6. A primer for reverse transcribing an isolated nucleic acid molecule of claim 1.

7. A cDNA-transcript of an isolated nucleic acid molecule of claim 1.

8. A set of primer pairs for amplifying a cDNA-transcript of claim 7.

9. A polynucleotide for detecting an isolated nucleic acid molecule of claim 1.

10. A cDNA-transcript of claim 7, hybridized to an isolated nucleic acid molecule of claim 1.

11. (canceled)

12. A method for treating a disease comprising the step of:
administering an effective amount of an isolated nucleic acid molecule comprising a nucleotide sequence selected from the group consisting of SEQ ID NO: 2, SEQ ID NO: 1, SEQ ID NO: 3 to SEQ ID NO: 37, a fragment thereof, and a nucleotide sequence with at least 90%, 94%, 96% or greater sequence identity thereto to a subject in need thereof.

13. A method for diagnosing and/or prognosing of a disease comprising the steps of:
determining an expression profile of at least one nucleic acid molecule, which is differentially expressed in the disease in a blood sample from a subject, and
(ii) comparing said expression profile to a reference, wherein the comparison of said expression profile to said reference allows for the diagnosis and/or prognosis of the disease,
wherein the nucleotide sequence of said at least one nucleic acid molecule is selected from the group consisting of SEQ ID NO: 2, SEQ ID NO: 1, SEQ ID NO: 3 to SEQ ID NO: 37, a fragment thereof, and a nucleotide sequence with at least 90%, 94%, 96% or greater sequence identity thereto.

14. (canceled)

15. (canceled)

16. A kit for diagnosing and/or prognosing a disease, comprising:
(a) means for determining an expression profile of at least one isolated nucleic acid molecule comprising a nucleotide sequence selected from the group consisting of SEQ ID NO: 2, SEQ ID NO: 1, SEQ ID NO: 3 to SEQ ID NO: 37, a fragment thereof, and a nucleotide sequence with at least 90%, 94%, 96% or greater sequence identity thereto, or of a complement thereof, which comprise
(i) one or more polynucleotides for detecting the isolated nucleic acid molecule or the complement thereof, and
(ii) a biochip, a RT-PCT system, a PCR-system, a flow cytometer, a Luminex system or a next generation sequencing system. and
(b) one or more reference expression profiles,
wherein the expression profile in (a) and the reference expression profiles in (b) are determined from the same at least one nucleic acid molecule in the same type of blood sample.

17. The kit of claim 16, wherein the one or more polynucleotides comprise
(i) a primer for reverse transcribing the at least one isolated nucleic acid molecule into a cDNA-transcript, and
(ii) a set of primer pairs for amplifying the cDNA-transcript.

18. The method of claim 12, wherein the disease is selected from the group consisting of Multiple sclerosis (MS) and Alzheimer's Disease (AD).

19. The method of claim 13, wherein the disease is selected from the group consisting of Multiple sclerosis (MS) and Alzheimer's Disease (AD).

20. The kit of claim 16, wherein the disease is selected from the group consisting of Multiple sclerosis (MS) and Alzheimer's Disease (AD).


SEQUENCE LISTING (69 sequences)

SEQ ID NO: 1 - RNA, 22 nt, Homo sapiens
auucgcugggaauucagccucu

SEQ ID NO: 2 - RNA, 22 nt, Homo sapiens
cccaaaccuugucuggacaugg

SEQ ID NO: 3 - RNA, 22 nt, Homo sapiens
ugucuagacaaggcuggggaaa

SEQ ID NO: 4 - RNA, 22 nt, Homo sapiens
agaggcugaauucccagugagu

SEQ ID NO: 5 - RNA, 22 nt, Homo sapiens
uguuuagcauccuguagccugc

SEQ ID NO: 6 - RNA, 23 nt, Homo sapiens
ccuggguuacacaaguucuacaa

SEQ ID NO: 7 - RNA, 22 nt, Homo sapiens
gagggucugacugucacuugga

SEQ ID NO: 8 - RNA, 22 nt, Homo sapiens
agguuguaggaugcuaaacaga

SEQ ID NO: 9 - RNA, 22 nt, Homo sapiens
uagguaggauagucaacauguc

SEQ ID NO: 10 - RNA, 22 nt, Homo sapiens
ucucauuggucaggccugaguc

SEQ ID NO: 11 - RNA, 23 nt, Homo sapiens
guaggacuuguguaacccaggga

SEQ ID NO: 12 - RNA, 23 nt, Homo sapiens
ucaugucugaaccaaugagagcc

SEQ ID NO: 13 - RNA, 21 nt, Homo sapiens
auaauguccaagcugagagcc

SEQ ID NO: 14 - RNA, 22 nt, Homo sapiens
uggcguuacugcagauaagggu

SEQ ID NO: 15 - RNA, 21 nt, Homo sapiens
gugugugcaccugugucuguc

SEQ ID NO: 16 - RNA, 22 nt, Homo sapiens
uacucauguuccugguguagca

SEQ ID NO: 17 - RNA, 22 nt, Homo sapiens
acuccagcaucucauacaccug

SEQ ID NO: 18 - RNA, 22 nt, Homo sapiens
acaacuaaucugacuggguuua

SEQ ID NO: 19 - RNA, 22 nt, Homo sapiens
ccagcaggugcagaauuccaca

SEQ ID NO: 20 - RNA, 22 nt, Homo sapiens
uuagcaucuggcacuauggacu

SEQ ID NO: 21 - RNA, 22 nt, Homo sapiens
agagagagugacugaggcuagc

SEQ ID NO: 22 - RNA, 20 nt, Homo sapiens
ucaaacucccuccaaggucu

SEQ ID NO: 23 - RNA, 23 nt, Homo sapiens
ugagcuagggauaucuccugaga

SEQ ID NO: 24 - RNA, 23 nt, Homo sapiens
cagucucaguuacucucccuggu

SEQ ID NO: 25 - RNA, 22 nt, Homo sapiens
aggguacggacgauuuggagcu

SEQ ID NO: 26 - RNA, 22 nt, Homo sapiens
ugagcaagugaaguauguggua

SEQ ID NO: 27 - RNA, 22 nt, Homo sapiens
agcagagcaaaacggaccaugc

SEQ ID NO: 28 - RNA, 21 nt, Homo sapiens
ugcccucccuucucagucaaa

SEQ ID NO: 29 - RNA, 22 nt, Homo sapiens
uuggccaggguaaaaaucaaac

SEQ ID NO: 30 - RNA, 21 nt, Homo sapiens
acagacgcagguacacacaga

SEQ ID NO: 31 - RNA, 21 nt, Homo sapiens
uggaauucugcaccuggugau

SEQ ID NO: 32 - RNA, 21 nt, Homo sapiens
ccacauacuucacuugcucag

SEQ ID NO: 33 - RNA, 22 nt, Homo sapiens
ugcugggcaguguccuccaagu

SEQ ID NO: 34 - RNA, 21 nt, Homo sapiens
uccaacaaaugugacuugcuc

SEQ ID NO: 35 - RNA, 21 nt, Homo sapiens
uuggaggaaggccuauucacu

SEQ ID NO: 36 - RNA, 21 nt, Homo sapiens
augguccauuuugcucugcuu

SEQ ID NO: 37 - RNA, 22 nt, Homo sapiens
aaaccuagucagauuaguucuc

SEQ ID NO: 38 - RNA, 58 nt, Homo sapiens
agaggcugaauucccagugagugccuuuugaguugcauucgcugggaauucagccucu

SEQ ID NO: 39 - RNA, 58 nt, Homo sapiens
cccaaaccuugucuggacauggacuugcaugguccaugucuagacaaggcuggggaaa

SEQ ID NO: 40 - RNA, 62 nt, Homo sapiens
uagguaggauagucaacaugucccuaauuggcugggggacauguugacucuccuaccugggc

SEQ ID NO: 41 - RNA, 56 nt, Homo sapiens
uguuuagcauccuguagccugcauccuacucuguagguuguaggaugcuaaacaga

SEQ ID NO: 42 - RNA, 59 nt, Homo sapiens
gagggucugacugucacuuggagcuaaaccagucuccaaguggccaucagacccucuuu

SEQ ID NO: 43 - RNA, 60 nt, Homo sapiens
ucaugucugaaccaaugagagccugagaaaugagcugcucucauuggucaggccugaguc

SEQ ID NO: 44 - RNA, 60 nt, Homo sapiens
guaggacuuguguaacccagggaccugcauagaugugccuggguuacacaaguucuacaa

SEQ ID NO: 45 - RNA, 64 nt, Homo sapiens
ugacugggaagucagagagaggcuaccugugaugucaggagccugcccucccuucucagucaaa

SEQ ID NO: 46 - RNA, 59 nt, Homo sapiens
uggcguuacugcagauaaggguauuuaggugucaaucccuuaucugcgguaaagucagg

SEQ ID NO: 47 - RNA, 64 nt, Homo sapiens
augguccauuuugcucugcuucugaaagcugaguacuuugcaagcagagcaaaacggaccaugc

SEQ ID NO: 48 - RNA, 64 nt, Homo sapiens
cagucucaguuacucucccugguacuuggaggucccagugccagagagagugacugaggcuagc

SEQ ID NO: 49 - RNA, 59 nt, Homo sapiens
uccaacaaaugugacuugcucugcugaguuugaccagagcaggucacauuuguuggaca

SEQ ID NO: 50 - RNA, 60 nt, Homo sapiens
gugugugcaccugugucugucuguauucguguuguauagacagacgcagguacacacaga

SEQ ID NO: 51 - RNA, 63 nt, Homo sapiens
ugagcuagggauaucuccugagaaugucuccucccuauucucaggaaaaaucccuagcuuggu

SEQ ID NO: 52 - RNA, 56 nt, Homo sapiens
aagaccuuggagggaguuuggugucaguuuucagcaucaaacucccuccaaggucu

SEQ ID NO: 53 - RNA, 64 nt, Homo sapiens; identical to SEQ ID NO: 47

SEQ ID NO: 54 - RNA, 59 nt, Homo sapiens
uuggaggaaggccuauucacuuagaagcaguuaccagugcugggcaguguccuccaagu

SEQ ID NO: 55 - RNA, 56 nt, Homo sapiens
ccagcaggugcagaauuccacaguucccuuaauuguggaauucugcaccuggugau

SEQ ID NO: 56 - RNA, 58 nt, Homo sapiens
auaauguccaagcugagagccaaaucaaguuauuggcucucaguuugggcauuacucg

SEQ ID NO: 57 - RNA, 59 nt, Homo sapiens; identical to SEQ ID NO: 54

SEQ ID NO: 58 - RNA, 64 nt, Homo sapiens; identical to SEQ ID NO: 48

SEQ ID NO: 59 - RNA, 58 nt, Homo sapiens
ugagcaagugaaguaugugguagcagugaaguaucuaccacauacuucacuugcucag

SEQ ID NO: 60 - RNA, 58 nt, Homo sapiens; identical to SEQ ID NO: 59

SEQ ID NO: 61 - RNA, 57 nt, Homo sapiens
uacucauguuccugguguagcaacgugugguauggcuacacagagaacaugagaaca

SEQ ID NO: 62 - RNA, 60 nt, Homo sapiens; identical to SEQ ID NO: 50

SEQ ID NO: 63 - RNA, 56 nt, Homo sapiens; identical to SEQ ID NO: 55

SEQ ID NO: 64 - RNA, 61 nt, Homo sapiens
aaaccuagucagauuaguucucccuggugagccuuggggacaacuaaucugacuggguuua

SEQ ID NO: 65 - RNA, 68 nt, Homo sapiens
uuggccaggguaaaaaucaaacccugugucucaggccacaacagaguuugauuuuacccuggcccggu

SEQ ID NO: 66 - RNA, 63 nt, Homo sapiens
acuccagcaucucauacaccuggaugauguucuugugguccaggguacggacgauuuggagcu

SEQ ID NO: 67 - RNA, 61 nt, Homo sapiens; identical to SEQ ID NO: 64

SEQ ID NO: 68 - RNA, 63 nt, Homo sapiens; identical to SEQ ID NO: 66

SEQ ID NO: 69 - RNA, 66 nt, Homo sapiens
uuagcaucuggcacuauggacucucaaauguuagcuucuggcaccauggaugcucagauguuagcg